(19) United States
(12) Patent Application Publication
(10) Pub. No.: US 2022/0259670 A1
(43) Pub. Date: Aug. 18, 2022

(54) KIT AND METHOD FOR ANALYZING SINGLE T CELLS

(71) Applicant: ST. JUDE CHILDREN'S RESEARCH HOSPITAL, Memphis, TN (US)

(72) Inventors: Pradyot DASH, Memphis, TN (US); Paul THOMAS, Memphis, TN (US)

(21) Appl. No.: 17/616,279

(22) PCT Filed: Jun. 30, 2020
(86) PCT No.: PCT/US2020/040218

§ 371 Date: Dec. 3, 2021

Related U.S. Application Data
(60) Provisional application No. 62/869,204, filed on Jul. 1, 2019.

(51) Int. Cl. C12Q 1/6886; C12Q 1/6806

(52) U.S. Cl. CPC C12Q 1/6886; C12Q 2600/16; C12Q 1/6806

(57) ABSTRACT

A kit and method for analyzing nucleic acid molecules encoding T cell receptor (TCR) a and β from individual T cells are disclosed. In particular, a method for analyzing individual T cells using high-throughput multiplex amplification and deep sequencing of nucleic acids encoding TCRαβ is provided.

This patent application claims the benefit of priority from U.S. Provisional Ser. No. 62/869,204 filed Jul. 1, 2019, the contents of which are incorporated herein by reference in their entirety.

### INTRODUCTION

This invention was made with government support under Grant Number AI107625 awarded by the National Institutes of Health. The government has certain rights in the invention.

### BACKGROUND

T cells are defined by a surface T cell receptor (TCR) that mediates recognition of pathogen-associated epitopes, generally via interactions with peptide-major histocompatibility complexes (pMHC). T cell receptors are generated by germline recombinase activating gene (RAG)-mediated rearrangements of the genomic TCR locus, a process termed V(D)J recombination. This process has the potential to generate a significant number of diverse TCRs, with estimates ranging from 10¹⁵ to as high as 10⁶¹ possible receptors that could be generated by recombination, although only a relatively small portion of these is thought to appear in any individual (˜10⁶-10⁸). In mammals, two types of TCRs are possible, αβ and γδ, and different species produce different ratios of cells bearing these receptors. In humans and mice, αβ T cells dominate, representing up to 90% of the T cell compartment.

The pool of T cells that recognizes a specific epitope expresses diverse TCRs. The size of these naïve precursor repertoires has been estimated for various epitopes by limiting dilution techniques and, more recently, by a tetramer-based magnetic enrichment approach, the latter of which finds pool sizes ranging between 50 and 500 naïve cells per epitope, on average. Due to the rounds of expansion that T cells undergo in the thymus during development, it has been assumed that there are multiple naïve cells with identical TCRs. However, sequencing the naïve repertoire of epitope-specific responses in mice has instead shown that most naïve cells contain a unique receptor, with a very low rate of duplicates among cells.

Sequencing the nucleic acids encoding the T cell receptor requires identifying the specific V-region used by the α or β chain and obtaining the complete sequence of the hypervariable CDR3 region, the site of RAG-mediated V(D)J junctional diversity. Due to the availability of TCR Vβ staining reagents in the human and mouse, analyses of the repertoire initially focused solely on the TCRβ chain. Subsequently, two broad approaches to sequencing the TCR repertoire have emerged: single-cell based methods that permit direct pairing of the α and β chains (Dash, et al. (2011) J. Clin. Invest. 121:288-295; Wang, et al. (2012) Sci. Transl. Med. 4:128ra42; Kim, et al. (2012) PLoS One 7:e37338; and Han, et al. (2014) Nat. Biotechnol. 32(7):684-692), and deep sequencing-based methods that amplify single chains from pools of cells (Robins, et al. (2009) Blood 114:4099-4107; Weinstein, et al. (2009) Science 324:807-810; Freeman, et al. (2009) Genome Res. 19:1817-1824) where pairing can be achieved through specific sort conditions and algorithmic imputation (Howie, et al. (2015) Sci. Transl. Med. 7:301ra131). Single cell multiplex techniques for TCRαβ or TCRγδ profiling have been described (Dash, et al. (2017) Nature 547(7661):89-92; Dash, et al. (2015) Meth. Mol. Biol. 1343:181-197; Guo, et al. (2016) Mol. Ther. Methods Clin. Dev. 3:15054; Guo, et al. (2018) Immunity 49(3):531-44; US 2019/0040381 A1). However, a large-scale multiplexing approach adapted to single cell deep sequencing is needed to increase the throughput of single cell TCR profiling.

### SUMMARY OF THE INVENTION

This invention provides a kit, which includes a first set of primers, wherein the first set of primers include (a) a first set of primers, wherein the first set of primers comprises: (i) a first set of forward primers comprising the nucleotide sequences of SEQ ID NOs:1-40 and SEQ ID NOs:42-70 having a length ranging from 15 to 40 nucleotides, (ii) a first set of reverse primers comprising the nucleotide sequences of SEQ ID NOs:41 and 71 having a length ranging from 15 to 40 nucleotides; and (b) a second set of primers, wherein the second set of primers comprises: (i) a second set of forward primers comprising the nucleotide sequences of SEQ ID NOs:72-111 and SEQ ID NO:130-158 having a length ranging from 40 to 70 nucleotides, and (ii) a second set of reverse primers comprising the nucleotide sequences of SEQ ID NOs:112-129 and SEQ ID NOs:159-176 having a length ranging from 50 to 90 nucleotides; or (c) a first set of primers, wherein the first set of primers comprises: (i) a first set of forward primers comprising the nucleotide sequences of SEQ ID NOs:177-199 and SEQ ID NOs:201-217 having a length ranging from 15 to 40 nucleotides, (ii) a first set of reverse primers comprising the nucleotide sequences of SEQ ID NOs:200 and 218 having a length ranging from 15 to 40 nucleotides; and (d) a second set of primers, wherein the second set of primers comprises: (i) a second set of forward primers comprising the nucleotide sequences of SEQ ID NOs:219-244 and SEQ ID NO:263-289 having a length ranging from 40 to 70 nucleotides, and (ii) a second set of reverse primers comprising the nucleotide sequences of SEQ ID NOs:245-262 and SEQ ID NOs:290-307 having a length ranging from 50 to 90 nucleotides.

A method for analyzing nucleic acid molecules encoding T cell receptor (TCR) α and β from single T cells is also provided, which includes the steps of (a) sorting single T cells from a sample comprising a plurality of T cells into separate locations; (b) amplifying nucleic acid molecules encoding TCR α and β from one or more single T cells using the first set of primers from the kit to produce a first set of amplicon products in one or more locations of the separate locations; (c) performing nested polymerase chain reaction (PCR) on the amplified nucleic acid molecules in the first set of amplicon products with the second set of primers from the kit to produce a second set of amplicon products; and (d) sequencing the second set of amplicon products. In one embodiment, the sequencing step (d) includes (i) carrying out a third round of PCR to produce a third set of amplicon products, and (ii) subjecting the third amplicon products to next generation sequencing. In certain embodiments, the target nucleic acids are mRNAs. In other embodiments, the sample is collected from a subject. In further embodiments, the performing step (c) includes dividing the first set of amplicon products into two pools; and performing nested PCR on the first pool and second pool separately, wherein (i) the first pool is amplified with the second set of forward primers having the nucleotide sequences of SEQ ID NOs:72-111, and the second set of reverse primers having the nucleotide sequences of SEQ ID NOs:112-129, and the second pool is amplified with the second set of forward primers having the nucleotide sequences of SEQ ID NOs:130-158, and the second set of reverse primers having the nucleotide sequences of SEQ ID NOs:159-176; or (ii) the first pool is amplified with the second set of forward primers having the nucleotide sequences of SEQ ID NOs:219-244, and the second set of reverse primers having the nucleotide sequences of SEQ ID NOs:245-262, and the second pool is amplified with the second set of forward primers having the nucleotide sequences of SEQ ID NOs:263-289, and the second set of reverse primers having the nucleotide sequences of SEQ ID NOs:290-307.

### BRIEF DESCRIPTION OF THE DRAWINGS

FIG. 1 shows a schematic of the method of this invention.

FIG. 2 demonstrates that the method of the invention provided increased paired data.

FIG. 3 shows the number of cells with one, two or three TCRα and TCRβ sequences per cell.

### DETAILED DESCRIPTION OF THE INVENTION

A multiplex panel of chimeric primer sequences designed to amplify TCRαβ sequences from single cells in an unbiased manner has now been developed. These primer sequences allow for the TCR repertoire to be analyzed using conventional next generation sequencing-based platforms in a highly efficient manner. Indeed, the instant single-cell TCRαβ amplification and sequencing strategy offers a highly transparent, cell number agnostic approach for processing up to 6300 cells at a time. Accordingly, this invention provides a kit and method for amplifying and analyzing TCRαβ sequences from single T cells using a multiplex panel of chimeric oligonucleotide sequences designed to amplify TCRαβ sequences in an unbiased manner. Using the kit and method of this invention, paired TCRαβ sequences can be isolated at the single cell level in response to a variety of immune responses such as viral infections, tumors, and autoimmune patients thereby allowing for the design of effective immune cell-based therapies.

The present disclosure provides kits containing oligonucleotide primers and methods for analyzing nucleic acids encoding TCRαβ from individual T cells by high-throughput multiplex amplification and sequencing of the nucleic acids encoding the TCRαβ. As used herein, the term “primer” or “oligonucleotide primer” refers to an oligonucleotide that hybridizes to the template strand of a nucleic acid and initiates synthesis of a nucleic acid strand complementary to the template strand when placed under conditions in which synthesis of a primer extension product is induced, i.e., in the presence of nucleotides and a polymerization-inducing agent such as a DNA or RNA polymerase and at suitable temperature, pH, metal concentration, and salt concentration. The primer is generally single-stranded for maximum efficiency in amplification, but may alternatively be double-stranded. If double-stranded, the primer can first be treated to separate its strands before being used to prepare extension products. This denaturation step is typically achieved by heat, but may alternatively be carried out using alkali, followed by neutralization. Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA or RNA synthesis.

The method of this invention generally involves sorting of single T cells into separate locations (e.g., separate wells of a multi-well titer plate) followed by nested polymerase chain reaction (PCR) amplification of nucleic acids encoding TCRs using the primers disclosed herein. The amplicons are barcoded to identify their cell of origin, combined, and analyzed by deep sequencing. Using the kit and method of the present disclosure, TCRs from individual T cells can be reconstituted for functional studies, ligand discovery, and/or screening therapeutics.

More specifically, this invention provides a kit and method for analyzing nucleic acid molecules encoding TCRαβ from individual T cells by sorting single T cells from a sample including a plurality of T cells into separate locations; amplifying nucleic acid molecules encoding TCRα and TCRβ from one or more single T cells using a first set of external primers to produce a first set of amplicon products in one or more locations of the separate locations; performing nested polymerase chain reaction (PCR) on the amplified nucleic acid molecules encoding TCRα and TCRβ in the first set of amplicon products with the unique forward and reverse nested primers of this disclosure to produce a second set of amplicon products, wherein the reverse nested primer includes a barcode sequence and the forward and reverse nested primers have adapters attached thereto; and sequencing the second set of amplicon products, e.g., via a third round of PCR and next generation sequencing. Using the method of this invention, a wide variety of diseases, including inflammatory disorders, autoimmune diseases, infectious diseases, and cancer can be diagnosed and treated.

In carrying out the method of this invention, a biological sample including T cells is collected from a subject. The biological sample can be any sample of bodily fluid or tissue containing T cells, including but not limited to, samples of blood, thymus, spleen, lymph nodes, bone marrow, a tumor biopsy, or an inflammatory lesion biopsy. In particular, samples of T cells may be taken from sites of inflamed, infected, or injured tissue, including but not limited to sites of tumors, transplant rejection, tissue damage, such as caused by traumatic injury or autoimmune disease, and organs or tissues targeted by pathogenic organisms. The biological sample may also include samples from in vitro cell culture resulting from the growth of T cells from the subject in culture. The biological sample can be obtained from a subject by conventional techniques. For example, blood can be obtained by venipuncture. Surgical techniques for obtaining solid tissue samples are well known in the art. Samples may be obtained from a subject prior to diagnosis and throughout a course of treatment.

Subsequently, single T cells are isolated from the biological sample and sorted into separate locations. The separate locations can be separate reaction containers, such as wells of a multi-well plate (e.g., a 96-well plate, 384-well plate, 1536-well plate) or microwell array, capillaries or tubes (e.g., 0.2 mL tubes, 0.5 mL tubes, 1.5 mL tubes), or chambers in a microfluidic device. Alternatively, the separate locations can be emulsion droplets that spatially separate cells.

Various methods are known in the art for isolating single cells. In some embodiments, the sample is sorted to obtain single T cells using a flow cytometer. Methods of preparing a sample of cells for flow cytometry analysis is described in, e.g., U.S. Pat. Nos. 5,378,633; 5,631,165; 6,524,858; 5,266,269; 5,017,497; 6,549,876; US 2012/0178098; US 2008/0153170; US 2001/0006787; US 2008/0158561; US 2010/0151472; US 2010/0099074; US 2010/0009364; US 2009/0269800; US 2008/0241820; US 2008/0182262; US 2007/0196870; US2008/0268494; WO 99/54494; Brown, et al. (2000) Clin. Chem. 46:1221-9; McCoy, et al. (2002) Hematol. Oncol. Clin. North Am. 16:229-43; and Scheffold (2000) J. Clin. Immunol. 20:400-7.

In some instances, single T cells can be isolated from a biological sample by appropriate dilution of a sample to allow distribution of a single cell in a small isolation volume to a separate location. In certain embodiments, a microfluidic device is used for isolating single cells and distributing single cells to separate locations in the device, such as separate wells or chambers. Alternatively, a microfluidic device can be used to generate emulsion droplets containing single cells. For a description of techniques for isolating single cells and microfluidic devices for sorting single cells, see, e.g., Huang, et al. (2014) Lab Chip. 14(7):1230-1245; Zare, et al. (2010) Annu. Rev. Biomed. Eng. 12:187-201; Novak, et al. (2011) Angew. Chem. Int. Ed. 50:390-395; US 2010/0255471; US 2010/0285975; US 2010/0021984; US 2010/0173394; WO 2009/145925; and US 2009/0181859.

“Microfluidics device” means an integrated system of one or more chambers, ports, and channels that are interconnected and in fluid communication and designed for carrying out an analytical reaction or process, either alone or in cooperation with an appliance or instrument that provides support functions, such as sample introduction, fluid and/or reagent driving means, temperature control, detection systems, data collection and/or integration systems, and the like. Microfluidics devices may further include valves, pumps, and specialized functional coatings on interior walls, e.g., to prevent adsorption of sample components or reactants, facilitate reagent movement by electroosmosis, or the like. Such devices are usually fabricated in or as a solid substrate, which may be glass, plastic, or other solid polymeric materials, and typically have a planar format for ease of detecting and monitoring sample and reagent movement, especially via optical or electrochemical methods. Features of a microfluidic device usually have cross-sectional dimensions of less than a few hundred square micrometers and passages typically have capillary dimensions, e.g., having maximal cross-sectional dimensions of from about 500 μm to about 0.1 μm. Microfluidics devices typically have, volume capacities in the range of from 1 μL to a few nL, e.g., 10-100 nL. The fabrication and operation of microfluidics devices are well-known in the art as described in U.S. Pat. Nos. 6,001,229; 5,858,195; 6,010,607; 6,033,546; 5,126,022; 6,054,034; 6,613,525; 6,399,952; WO 02/24322; WO 99/19717; and U.S. Pat. Nos. 5,587,128; 5,498,392.

In certain embodiments, the sample is labeled with one or more detectable labels that bind to cells within the sample before sorting the cells. The terms “label” and “detectable label” refer to a molecule capable of detection, including, but not limited to, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like. In some cases, the detectable label is linked to a binding agent that binds to a binding partner on a T cell in the sample. In case of labeling T cells, the binding agent may be an antibody (e.g., anti-CD3, anti-CD4, anti-CD8, anti-αβTCR, anti-CD14, anti-CD25, anti-CD45RA, anti-CD45RO, anti-FOXP3, etc.) or major histocompatibility complex (MHC) tetramer that specifically binds to a binding partner on or in a T cell. Thus, in some cases, the T cell is permeabilized before labeling. In some embodiments, one or more detectable labels is used to classify a cell, e.g., T cell, within a sample, based on the amount of label bound to the cell.

In some embodiments, a subset of cells within a sample is sorted as single cells into separate locations. Thus, cells may be sorted to include a first subset and exclude a second subset of cells within the sample. The first subset and second subset may be defined by a number of factors, including, but not limited to, amount of detectable label that is bound, size, light scattering properties, amount of staining by dyes that indicate viability or lack thereof, etc., of a cell. Thus, in some embodiments, a T cell that is labeled with an anti-CD8, anti-CD14, MI-IC tetramer or a combination thereof, and is not labeled as being dead, is included to be sorted to generate single T cells in separate locations.

In some cases, sorting the T cells into separate locations as single cells may result in a subset of the separate locations having two or more T cells. These locations with potentially more than one T cells may be identified and flagged during data analysis of the sequencing data, and data from such locations in some cases may be removed from further analysis.

As explained above, the primers described herein may be used in polymerase chain reaction (PCR)-based techniques, such as RT-PCR, for amplification of T cell mRNA. As is conventional in the art, “polymerase chain reaction,” or “PCR” means a reaction for the in vitro amplification of specific DNA sequences by the simultaneous primer extension of complementary strands of DNA. In PCR, a pair of primers is employed in excess to hybridize to the complementary strands of the target nucleic acid. The primers are each extended by a polymerase using the target nucleic acid as a template. The extension products become target sequences themselves after dissociation from the original target strand. New primers are then hybridized and extended by a polymerase, and the cycle is repeated to geometrically increase the number of target sequence molecules. The PCR method for amplifying target nucleic acid sequences in a sample is well-known in the art and has been described in, e.g., Innis, et al. (eds.)(1990) PCR Protocols, Academic Press, NY; Taylor (1991) Polymerase chain reaction: basic principles and automation, in PCR: A Practical Approach, McPherson et al. (eds.) IRL Press, Oxford; Saiki, et al. (1986) Nature 324:163; as well as in U.S. Pat. Nos. 4,683,195; 4,683,202; and 4,889,818.

In particular, PCR uses relatively short oligonucleotide primers that flank the target nucleotide sequence to be amplified, oriented such that their 3′ ends face each other, each primer extending toward the other. The polynucleotide sample is extracted and denatured, e.g., by heat, and hybridized with first and second primers that are present in molar excess. Polymerization is catalyzed in the presence of the four deoxyribonucleotide triphosphates (dNTPs: dATP, dGTP, dCTP and dTTP) using a primer- and template-dependent polynucleotide polymerizing agent, such as any enzyme capable of producing primer extension products, for example, E. coli DNA polymerase I, Kienow fragment of DNA polymerase I, T4 DNA polymerase, thermostable DNA polymerases isolated from Thermus aquaticus (Tag), available from a variety of sources (for example, Perkin Elmer), Thermus thermophilus (United States Biochemicals), Bacillus stereothermophilus (Bio-Rad), or Thermococcus litoralis (“Vent” polymerase, New England Biolabs). This results in two “long products” which contain the respective primers at their 5′ ends covalently linked to the newly synthesized complements of the original strands. The reaction mixture is then returned to polymerizing conditions, e.g., by lowering the temperature, inactivating a denaturing agent, or adding more polymerase, and a second cycle is initiated. The second cycle provides the two original strands, the two long products from the first cycle, two new long products replicated from the original strands, and two “short products” replicated from the long products. The short products have the sequence of the target sequence with a primer at each end. On each additional cycle, an additional two long products are produced, and a number of short products equal to the number of long and short products remaining at the end of the previous cycle. Thus, the number of short products containing the target sequence grows exponentially with each cycle. In some cases, PCR is carried out with a commercially available thermal cycler, e.g., Perkin Elmer.

RNA may be amplified by reverse transcribing the RNA into cDNA, and then performing PCR. This type of amplification, referred to as “reverse transcription PCR,” or “RT-PCR,” is well-known in the art and described, e.g., in U.S. Pat. No. 5,168,038, which is incorporated herein by reference. Alternatively, a single enzyme may be used for both steps as described in U.S. Pat. No. 5,322,770, incorporated herein by reference in its entirety. RNA may also be reverse transcribed into cDNA, followed by asymmetric gap ligase chain reaction (RT-AGLCR) as described by Marshall, et al. (1994) PCR Meth. App. 4:80-84. Suitable DNA polymerases include reverse transcriptases, such as avian myeloblastosis virus (AMV) reverse transcriptase (available from, e.g., Seikagaku America, Inc.) and Moloney murine leukemia virus (MMLV) reverse transcriptase (available from, e.g., Bethesda Research Laboratories).

Promoters or promoter sequences suitable for incorporation in the primers are nucleic acid sequences (either naturally occurring, produced synthetically or a product of a restriction digest) that are specifically recognized by an RNA polymerase that recognizes and binds to that sequence and initiates the process of transcription whereby RNA transcripts are produced. The sequence may optionally include nucleotide bases extending beyond the actual recognition site for the RNA polymerase which may impart added stability or susceptibility to degradation processes or increased transcription efficiency. Examples of useful promoters include those which are recognized by certain bacteriophage polymerases such as those from bacteriophage T3, T7 or SP6, or a promoter from E. coli. These RNA polymerases are readily available from commercial sources, such as New England Biolabs and Epicentre.

Some of the reverse transcriptases suitable for use in the methods herein have an RNAse H activity, such as AMV reverse transcriptase. In some cases, exogenous RNAse H, such as E. coli RNAse H, is added, even when AMV reverse transcriptase is used. RNAse H is readily available from, e.g., Bethesda Research Laboratories.

The RNA transcripts produced by these methods may serve as templates to produce additional copies of the target sequence through the above-described mechanisms. The system is autocatalytic and amplification occurs autocatalytically without the need for repeatedly modifying or changing reaction conditions such as temperature, pH, ionic strength or the like.

The method of the present disclosure uses a multiplexed nested PCR approach. “Nested PCR” refers to PCR that is carried out in at least two steps, wherein the amplicon product from a first round of PCR becomes the template for a second round of PCR using a second set of primers, at least one of which binds to an interior location of the amplicon from the first round of PCR, to generate a second amplicon product. In certain embodiments, a third round of PCR is carried out on the second amplicon product using a third set of primers to generate a third amplicon product, which is sequenced.

In certain embodiments, the nested PCR is multiplexed, wherein the nested PCR is carried out with T cell target sequences encoding TCRs simultaneously in the same reaction mixture. See, e.g., Bernard, et al. (1999) Anal. Biochem. 273:221-226. Distinct sets of primers are employed for each sequence being amplified as described herein. Exemplary primers (SEQ ID NOs:1-307) are provided in Tables 1-8 for amplifying human and mouse TCRs (e.g., both α and β chains of the heterodimer), and also for adding barcodes and sequencing adapters for paired-end sequencing. Changes to the nucleotide sequences of these primers may be introduced corresponding to genetic variations in particular T cells. For example, up to three nucleotide changes, including 1 nucleotide change, 2 nucleotide changes, or three nucleotide changes, may be made in a sequence selected from the group of SEQ ID NOs: 1-307, wherein the oligonucleotide primer is capable of hybridizing to and amplifying or sequencing a T cell target nucleic acid (i.e., nucleic acids encoding TCRαβ).

In certain cases, a first set of primers used to amplify a target nucleic acid, i.e., a nucleic acid encoding TCRαβ, may contain a primer that specifically hybridizes to and amplifies, when paired with another appropriate primer in the first set, the target nucleic acid during a first round of PCR. A second set of primers may then be used to further amplify the target nucleic acid when the second set contains a primer that specifically hybridizes to and amplifies, when paired with another appropriate primer in the second set, a specific amplification product of the first round of PCR during a second round of PCR. Similarly, a third set of primers may then be used to further amplify the target nucleic acid when the third set contains a primer that specifically hybridizes to and amplifies, when paired with another appropriate primer in the third set, a specific amplification product of the second round of PCR during a third round of PCR.

In some embodiments, primers within a set of primers may include, in addition to a sequence that hybridizes to a target nucleic acid, or an amplification product thereof, a common sequence and/or a barcode sequence. The common sequence may be the same sequence among a plurality of primers that otherwise hybridize to and amplify, when appropriately paired with another primer, different target nucleic acids, or amplification products thereof. In some embodiments, the common sequence in a primer used during a round of PCR enables a primer used during a following round of PCR to anneal to and amplify, when paired with an appropriate primer, the target nucleic acid by serving as an annealing site for the primer used during a following round of PCR. As such, in some cases, the common sequence in a primer used during a round of PCR is a sequence that does not hybridize to target-specific sequences of a target nucleic acid, or to a specific amplification product from a previous round of PCR. In some cases, the common sequence is a sequence that hybridizes to a target nucleic acid, if, for example, the target nucleic acid includes a sequence that is shared among different target nucleic acids, e.g., a sequence encoding a constant region of a TCR.

The multiplexed PCR reactions may be carried out in one or more of the separate locations into which single T cells from a sample have been sorted. Ideally, the amplification products of the multiplexed PCR reaction, which are in multiple separate locations, are combined into one pool before sequencing. In certain embodiments, the barcode sequence used in one of the rounds of the multiplexed PCR reactions may be used to enable identification of the location, e.g., well, from which a particular sequenced amplification product originated, as described further herein.

The present disclosure provides sets of primers that amplify nucleic acid molecules encoding T cell receptors, or a portion thereof. In aspects pertaining to amplification of nucleic acid molecules encoding human T cell receptors, a first set of primers includes a first set of forward primers set forth in SEQ ID NOs:1-40 and SEQ ID NOs:42-70, or a variant thereof that differs by up to three nucleotides, and a first set of reverse primers set forth in SEQ ID NOs:41 and 71, or a variant thereof that differs by up to three nucleotides, wherein the first set of primers amplify nucleic acid molecules encoding T cell receptors, or a portion thereof, from single T cells to produce a first set of amplicons. In aspects pertaining to amplification of nucleic acid molecules encoding mouse T cell receptors, a first set of primers includes a first set of forward primers set forth in SEQ ID NOs:177-199 and SEQ ID NOs:201-217, or a variant thereof that differs by up to three nucleotides, and a first set of reverse primers set forth in SEQ ID NOs:200 and 218, or a variant thereof that differs by up to three nucleotides, wherein the first set of primers amplify nucleic acid molecules encoding T cell receptors, or a portion thereof, from single T cells to produce a first set of amplicons. The T cell receptors amplified with the first set of primers include the T cell receptor alpha chain and T cell receptor beta chain.

The present disclosure further provides a second set of nested primers that amplify the first set of amplicons. In aspects pertaining to amplification of nucleic acid molecules encoding human T cell receptors, a second set of nested primers includes a second set of forward nested primers set forth in SEQ ID NOs:72-111 and SEQ ID NOs:130-158, or a variant thereof that differs by up to three nucleotides, and a second set of reverse nested primers set forth in SEQ ID NOs:112-129 and SEQ ID NOs:159-176, or a variant thereof that differs by up to three nucleotides, wherein the second set of nested primers amplify the first set of amplicons to produce a second set of amplicons. In aspects pertaining to amplification of nucleic acid molecules encoding mouse T cell receptors, a second set of nested primers includes a second set of forward nested primers set forth in SEQ ID NOs:219-244 and SEQ ID NOs:263-289, or a variant thereof that differs by up to three nucleotides, and a second set of reverse nested primers set forth in SEQ ID NOs:245-262 and SEQ ID NOs:290-307, or a variant thereof that differs by up to three nucleotides, wherein the second set of nested primers amplify the first set of amplicons to produce a second set of amplicons

Advantageously, barcode sequences are included in the second set of reverse nested primers to identify the single T cell from which each amplified nucleic acid originated. The use of barcodes allows nucleic acid analytes from different cells to be pooled in a single reaction mixture for sequencing while still being able to trace back a particular target nucleic acid to the particular cell from which it originated. Each cell is identified by a unique barcode sequence comprising at least six nucleotides. In accordance with this invention, barcode sequences are added during amplification by carrying out PCR with a primer that contains a region comprising the barcode sequence and a region that is complementary to the target nucleic acid of interest such that the barcode is incorporated into the final amplified nucleic acid product. Exemplary barcode sequences include, but are not limited to, 5′-ATCACG-3′, 5′-CGATGT-3′, 5′-TTAGGC-3′, 5′-TGACCA-3′, 5′-GCCAAT-3′, 5′-CAGATC-3′, 5′-ACTTGA-3′, 5′-GATCAG-3′, 5′-TAGCTT-3′, 5′-GGCTAC-3′, 5′-CTTGTA-3′, 5′-ACAGTG-3′, 5′-ACAGTG-3′, 5′-AGTCAA-3′, 5′-AGTTCC-3′, 5′-ATGTCA-3′, 5′-CCGTCC-3′, 5′-GTAGAG-3′, 5′-GTCCGC-3′, 5′-CGTGAT-3′, 5′-ACATCG-3′, 5′-GCCTAA-3′, 5′-TGGTCA-3′, 5′-ATTGGC-3′, 5′-GATCTG-3′, 5′-TCAAGT-3′, 5′-CTGATC-3′, 5′-AAGCTA-3′, 5′-GTAGCC-3′, 5′-TACAAG-3′, 5′-CACTGT-3′, 5′-TTGACT-3′, 5′-GGAACT-3′, 5′-TGACAT-3′, 5′-GGACGG-3′, 5′-CTCTAC-3′, OR 5′-GCGGAC-3′. See ILLUMINA® (2015) Illumina Adapter Sequences, Document #1000000002694 v00. In certain embodiments, single cells are initially sorted to separate locations in an ordered array or multi-well plate where the cell can be identified by its position using barcodes. In certain embodiments, a barcode sequence is added to one end of an amplicon to identify the position of a cell in a multi-well plate. Exemplary primers for introducing barcodes into an amplicon are provided in Tables 3-4 and 7-8. In one embodiment, a primer for introducing a barcode sequence into an amplicon of a nucleic acid encoding a TOR has a sequence set forth in SEQ ID NOs:112-129 and SEQ ID NOs:159-176; or SEQ ID NOs:245-262 and SEQ ID NOs:290-307.

In addition to barcodes, adapter sequences are added to amplicons to facilitate high-throughput amplification or sequencing. For example, a pair of adapter sequences are added at the 5′ and 3′ ends of a DNA template to allow amplification and/or sequencing of multiple DNA templates simultaneously by the same set of primers. Exemplary adapter sequences include the sequences: TCGTCGGCAGCGTCAGATGTGTATAAGAGACAG (SEQ ID NO:308) and GTCTCGTGGGCTCGGAGATGTGTATAAGAGACAG (SEQ ID NO:309). See ILLUMINA®, 16S Metagenomic Sequencing Library Preparation, Preparing 16S Ribosomal RNA Gene Amplicons for the Illumina MiSeq System, Part #15044223 Rev. B. Primers incorporating these adapter sequences are set forth in SEQ ID NOs:112-129 and SEQ ID NOs:159-176 (Tables 3-4) and SEQ ID NOs:245-262 and SEQ ID NOs:290-307 (Tables 7-8).

In some embodiments, the first set of forward primers and/or the first set of reverse primers, as described above, do not include a barcode sequence and/or an adapter sequence. In other embodiments, the second set of forward nested primers and/or the second set of reverse nested primers, as described above, include a barcode sequence and/or an adapter sequence.

The primers of this invention can be readily synthesized by standard techniques, e.g., solid phase synthesis via phosphoramidite chemistry, as disclosed in U.S. Pat. Nos. 4,458,066; 4,415,732; Beaucage, et al. (1992) Tetrahedron 48:2223-2311. Other chemical synthesis methods include, for example, the phosphotriester method described by Narang, et al. (1979) Meth. Enzymol. 68:90 and the phosphodiester method disclosed by Brown, et al. (1979) Meth. Enzymol. 68:109. Poly(A) or poly(C), or other non-complementary nucleotide extensions may be incorporated into oligonucleotides using these same methods. Hexaethylene oxide extensions may be coupled to the oligonucleotides by methods known in the art. See, e.g., Cload, et al. (1991) J. Am. Chem. Soc. 113:6324-6326; U.S. Pat. No. 4,914,210; Durand, et al. (1990) Nucleic Acids Res. 18:6353-6359; and Horn, et al. (1986) Tet. Lett. 27:4705-4708.

The primers of this invention are in the range of between 10-100 nucleotides in length, such as 15-40, 20-40, 15-70, 50-90 and so on, more typically in the range of between 15-90 nucleotides long, and any length between the stated ranges. In certain embodiments, a primer oligonucleotide has a sequence selected from the group of SEQ ID NOs:1-307 or a fragment thereof including at least about 6 contiguous nucleotides, at least about 8 contiguous nucleotides, at least about 10-12 contiguous nucleotides, or at least about 15-20 contiguous nucleotides; or a variant thereof with a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto. Changes to the nucleotide sequences of SEQ ID NOs:1-307 may be introduced corresponding to genetic variations in particular T cells. In certain embodiments, up to three nucleotide changes, including 1 nucleotide change, 2 nucleotide changes, or three nucleotide changes, may be made in a sequence selected from the group of SEQ ID NOs:1-307, wherein the oligonucleotide primer is capable of hybridizing to and amplifying a particular T cell receptor target nucleic acid.

Moreover, the oligonucleotides, particularly the primer oligonucleotides for amplification or sequencing, may be coupled to labels for detection. There are several means known for derivatizing oligonucleotides with reactive functionalities which permit the addition of a label. For example, several approaches are available for biotinylating probes so that radioactive, fluorescent, chemiluminescent, enzymatic, or electron dense labels can be attached via avidin. See, e.g., Broken, et al. (1978) Nucl. Acids Res. 5:363-384, which discloses the use of ferritin-avidin-biotin labels; and Chollet, et al. (1985) Nucl. Acids Res. 13:1529-1541, which discloses biotinylation of the 5′ termini of oligonucleotides via an aminoalkylphosphoramide linker arm. Several methods are also available for synthesizing amino-derivatized oligonucleotides which are readily labeled by fluorescent or other types of compounds derivatized by amino-reactive groups, such as isothiocyanate, N-hydroxysuccinimide, or the like, see, e.g., Connolly (1987) Nucl. Acids Res. 15:3131-3139; Gibson, et al. (1987) Nucl. Acids Res. 15:6455-6467 and U.S. Pat. No. 4,605,735. Methods are also available for synthesizing sulfhydryl-derivatized oligonucleotides, which can be reacted with thiol-specific labels, see, e.g., U.S. Pat. No. 4,757,141; Connolly, et al. (1985) Nucl. Acids Res. 13:4485-4502 and Spoat, et al. (1987) Nucl. Acids Res. 15:4837-4848. A comprehensive review of methodologies for labeling DNA fragments is provided in Matthews, et al. (1988) Anal. Biochem. 169:1-25.

For example, oligonucleotides may be fluorescently labeled by linking a fluorescent molecule to the non-ligating terminus of the molecule. Guidance for selecting appropriate fluorescent labels can be found in Smith, et al. (1987) Meth. Enzymol. 155:260-301; Karger, et al. (1991) Nucl. Acids Res. 19:4955-4962; and Guo, et al. (2012) Anal. Bioanal. Chem. 402(10):3115-3125. Fluorescent labels include fluorescein and derivatives thereof, such as disclosed in U.S. Pat. No. 4,318,846 and Lee, et al. (1989) Cytometry 10:151-164. Dyes for use in the present invention include 3-phenyl-7-isocyanatocoumarin, acridines, such as 9-isothiocyanatoacridine and acridine orange, pyrenes, benzoxadiazoles, and stilbenes, such as disclosed in U.S. Pat. No. 4,174,384. Additional dyes include Yakima Yellow, Texas Red, 3-(ε-carboxypentyl)-3′-ethyl-5,5′-dimethyloxa-carbocyanine (CYA), 6-carboxy fluorescein (FAM), 5,6-carboxyrhodamine-110 (R110), 6-carboxyrhodamine-6G (R6G), N′,N′,N′,N′-tetramethyl-6-carboxyrhodamine (TAMRA), 6-carboxy-X-rhodamine (ROX), 2′,4′,5′,7′,-tetrachloro-4-7-dichlorofluorescein (TET), 2′,7′-dimethoxy-4′,5′-6 carboxyrhodamine (JOE), 6-carboxy-2′,4,4′,5′,7,7′-hexachlorofluorescein (HEX), Dragonfly orange, ATTO-Tec, Bodipy, and VIC, and dyes available under the trademarks SYBR® green, SYBR® gold, CAL FLUOR® Orange 560, CAL FLUOR® Red, QUASAR® Blue 670, ALEXA®, Cy3®, and Cy5®. These dyes are commercially available from various suppliers such as Life Technologies (Carlsbad, Calif.), Biosearch Technologies (Novato, Calif.), and Integrated DNA Technologies (Coralville, Iowa). Fluorescent labels include fluorescein and derivatives thereof, such as disclosed in U.S. Pat. No. 4,318,846 and Lee, et al. (1989) Cytometry 10:151-164, and 6-FAM, JOE, TAMRA, ROX, HEX-1, HEX-2, ZOE, TET-1 or NAN-2, and the like.

Oligonucleotides can also be labeled with a minor groove binding (MOB) molecule, such as disclosed in U.S. Pat. Nos. 6,884,584; 5,801,155; Afonina, et al. (2002) Biotechniques 32:940-944, 946-949; Lopez-Andreo, et al. (2005) Anal. Biochem. 339:73-82; and Belousov, et al. (2004) Hum. Genomics 1:209-217. Oligonucleotides having a covalently attached MOB are more sequence specific for their complementary targets than unmodified oligonucleotides. In addition, an MOB group increases hybrid stability with complementary DNA target strands compared to unmodified oligonucleotides, allowing hybridization with shorter oligonucleotides.

Additionally, oligonucleotides can be labeled with an acridinium ester (AE) using the techniques described below. Current technologies allow the AE label to be placed at any location within the probe. See, e.g., Nelson, et al. (1995) “Detection of Acridinium Esters by Chemiluminescence” in Nonisotopic Probing, Blotting and Sequencing, Kricka L. J(ed) Academic Press, San Diego, Calif.; Nelson, et al. (1994) “Application of the Hybridization Protection Assay (HPA) to PCR” in The Polymerase Chain Reaction, Mullis et al. (eds.) Birkhauser, Boston, Mass.; Weeks, et al. (1983) Clin. Chem. 29:1474-1479; Berry, et al. (1988) Clin. Chem. 34:2087-2090. An AE molecule can be directly attached to the probe using non-nucleotide-based linker arm chemistry that allows placement of the label at any location within the probe. See, e.g., U.S. Pat. Nos. 5,585,481 and 5,185,439.

T cells may be pre-treated in any number of ways prior to amplification and sequencing of nucleic acids. For instance, in certain embodiments, the T cell may be treated to disrupt (or lyse) the cell membrane, for example by treating the samples with one or more detergents and/or denaturing agents (e.g., guanidinium agents). Nucleic acids may also be extracted from samples, for example, after detergent treatment and/or denaturing as described above. Total nucleic acid extraction may be performed using known techniques, for example by non-specific binding to a solid phase (e.g., silica). See, e.g., U.S. Pat. Nos. 5,234,809; 6,849,431; 6,838,243; 6,815,541; and 6,720,166.

In certain embodiments, the target nucleic acids are separated from non-homologous nucleic acids using capture oligonucleotides immobilized on a solid support. Such capture oligonucleotides contain nucleic acid sequences that are complementary to a nucleic acid sequence present in the target T cell nucleic acid analyte such that the capture oligonucleotide can “capture” the target nucleic acid. Capture oligonucleotides can be used alone or in combination to capture T cell nucleic acids. For example, multiple capture oligonucleotides can be used in combination, e.g., 2, 3, 4, 5, 6, etc. different capture oligonucleotides can be attached to a solid support to capture target T cell nucleic acids. In certain embodiments, one or more capture oligonucleotides can be used to bind T cell target nucleic acids either prior to or after amplification by primer oligonucleotides and/or sequencing.

As T cells may be sorted into single T cells in separate locations, e.g., separate wells, in the present method, as described above, some embodiments of the present disclosure include a composition including one or more sets of forward and reverse primers and/or sets of primer pairs, as described above, and nucleic acids from a single T cell. After single T cells are sorted to separate locations, they may be lysed in order to release cellular contents, such as nucleic acids (e.g., mRNA, miRNA, chromosomal DNA, mitochondrial DNA, etc.). The released nucleic acids may then provide templates, including any target nucleic acids, from which PCR may be carried out using the primer compositions of the present disclosure. A composition that contains nucleic acids from a single T cell may be distinguished from a composition that contains nucleic acids from two or more T cells by, e.g., determining the number of one or more autosomal loci of chromosomal DNA using sequencing or other suitable methods, as described in, e.g., Kalisky, et al. (2011) Nat. Methods 8:311; Fu, et al. (2011) Proc. Natl. Acad. Sci. USA 108:9026; and Shuga, et al. (2013) Nucleic Acids Res. 41:e159. Thus, in some embodiments, the composition contains one or more sets of forward and reverse primers and/or sets of primer pairs, as described above, and T cell nucleic acids from less than two T cells. In some embodiments, the composition contains no nucleases and/or contains nuclease inhibitors and/or provides buffering conditions that inhibits or reduces nucleic acid degradation at least until the first round of amplification.

Any high-throughput technique for sequencing can be used in the practice of the invention. DNA sequencing techniques include sequencing by synthesis using reversibly terminated labeled nucleotides, pyrosequencing, 454 sequencing, sequencing by synthesis using allele specific hybridization to a library of labeled clones followed by ligation, real-time monitoring of the incorporation of labeled nucleotides during a polymerization step, polony sequencing, SOLID sequencing, and the like. These sequencing approaches can thus be used to sequence target TCR nucleic acids of amplified from single T cells.

Certain high-throughput methods of sequencing include a step in which individual molecules are spatially isolated on a solid surface where they are sequenced in parallel. Such solid surfaces may include nonporous surfaces (such as in Solexa sequencing, e.g., Bentley, et al. (2008) Nature 456:53-59; or Complete Genomics sequencing, e.g., Drmanac, et al. (2010) Science 327:78-81), arrays of wells, which may include bead- or particle-bound templates (such as with 454, e.g. Margulies, et al. (2005) Nature 437:376-380; or Ion Torrent sequencing, e.g., US 2010/0137143 or US 2010/0304982), micromachined membranes (such as with SMRT sequencing, e.g., Rid, et al. (2009) Science 323:133-138), or bead arrays (as with SOLiD sequencing or polony sequencing, e.g., Kim, et al. (2007) Science 316:1481-1414). Such methods may include amplifying the isolated molecules either before or after they are spatially isolated on a solid surface. Prior amplification may include emulsion-based amplification, such as emulsion PCR, or rolling circle amplification. In certain embodiments, amplification is carried out using a third set of primers in a third round of PCR with the second amplicon product as a template, i.e., for paired-end sequencing. Of particular interest is sequencing on the ILLUMINA® MiSeq platform, which uses reversible-terminator sequencing by synthesis technology (see, e.g., Shen, et al. (2012) BMC Bioinformatics 13:160; Junemann, et al. (2013) Nat. Biotechnol. 31(4):294-296; Glenn (2011) Mol. Ecol. Resour. 11(5):759-769; Thudi, et al. (2012) Brief Funct. Genomics 11(1):3-11).

The present disclosure also provides for analyzing multiplexed single cell sequencing data, such as those acquired using the method of analyzing single T cells described herein. In one implementation, a user may access a file on a computer system, wherein the file is generated by sequencing multiplexed PCR amplification products from multiple single T cells by, e.g., a method of analyzing single T cells, as described herein. Thus, the file may include a plurality of sequencing reads for a plurality of nucleic acids derived from multiple T cells. Each of the sequencing reads may be a sequencing read of a nucleic acid that contains a target nucleic acid nucleotide sequence (i.e., a nucleotide sequence encoding T cell receptor) and one or more barcode sequences that identifies the single cell source (e.g., a single cell in a well in a multi-well plate, a capillary, a microfluidic chamber, etc.) from which the nucleic acid originated (e.g., after multiple nested PCR of the target nucleic acid expressed by a single T cell in the well). In some embodiments, the sequencing read is a paired-end sequencing read.

The sequencing reads in the file may be assembled to generate a consensus sequence of a target nucleic acid nucleotide sequence by matching the nucleotide sequence corresponding to the target nucleic acid sequence and the barcode sequences contained in each sequencing read. Those sequencing reads that originate from the same single cell source (e.g., same well) and have a target sequence that has a higher identity to a reference sequence than a threshold identity level may be assigned to the same target nucleic acid that was initially amplified from the single cell source, and may be grouped into a subset representing the target nucleic acid. The number of sequencing reads within the subset indicates how likely it is that the consensus sequence assembled from the sequencing reads in a subset is part of an actual nucleic acid molecule that was present in the single cell source. Thus, if the number of sequencing reads in a subset is above a background level, the consensus sequence derived from the subset may be considered to represent an actual sequence of a target nucleic acid in the single cell source. The consensus sequence may then be outputted, e.g., to a display, printout, database, etc.

In some embodiments, the reference sequence is a sequence for the target nucleic acid in a reference database, such as GENBANK®. Thus, in some embodiments, a target sequence in a first sequencing read in a subset of sequencing reads, as described above, is 80% or more, e.g., 85% or more, 90% or more, 95% or more, or up to 100% identical to a reference sequence for the target nucleic acid from a reference database. In some embodiments, the reference sequence is one or more other sequences in sequencing reads of the same subset. Thus, in such cases, a target nucleotide sequence in a first sequencing read in a subset of sequencing reads, as described above, is 80% or more, e.g., 85% or more, 90% or more, 95% or more, or up to 100% identical to a target nucleotide sequence in a second sequencing read in the same subset. In some instances, a target nucleotide sequence in a first sequencing read in a subset is 80% or more, e.g., 85% or more, 90% or more, 95% or more, or up to 100% identical to a target nucleotide sequence in all other sequencing reads in the same subset.

In certain embodiments, the sequencing reads are generated by a method of analyzing a T cell as disclosed herein. As such, in some embodiments, the target nucleic acid sequence contained in the sequenced nucleic acid is flanked on the 5′ and 3′ ends by a common sequence and optionally a barcode sequence. The barcode sequence may contain a sequence that specifies the single cell source of the target nucleic acid (e.g., the plate among a plurality of plates, the row among a plurality of rows in a multiwall plate, and/or the column among a plurality of columns in a multiwall plate, etc.). The common sequence and/or barcode sequence is incorporated into the amplified target nucleic acid during a round of the multiplex amplification process, e.g., during the second round of amplification, as described above, to provide for a primer annealing site that may be used in the next round, e.g., third round, of amplification. Thus, the common sequences at the ends of the amplified target nucleotide sequence may be sequences exogenous to the target nucleic acid, are ideally different from one another, and may not be a sequence that can hybridize to the target nucleotide sequence before the second round of amplification. The length of the common sequences may be in the range of 17 to 30 nucleotides long, e.g., 18 to 28 nucleotides long, 19 to 26 nucleotides long, including 20 to 25 nucleotides long.

The output of the analysis may be provided in any convenient form. In some embodiments, the output is provided on a user interface, a print out, in a database, etc. and the output may be in the form of a table, graph, raster plot, heat map etc. In some embodiments, the output is further analyzed to determine properties of the single cell from which a target nucleotide sequence was derived. Further analysis may include correlating expression of a plurality of target nucleotide sequences within single cells, principle component analysis, clustering, statistical analyses, etc.

A computer system for implementing the present computer-implemented method may include any arrangement of components as is commonly used in the art. The computer system may include a memory, a processor, input and output devices, a network interface, storage devices, power sources, and the like. The memory or storage device may be configured to store instructions that enable the processor to implement the present computer-implemented method by processing and executing the instructions stored in the memory or storage device.

In certain alternative embodiments, the present method of analyzing T cells includes stimulating T cells in a sample obtained from a subject before sorting single T cells into separate locations. The stimulating may be achieved by any convenient method. Stimulating T cells may include, but are not limited to, contacting the T cells with 12-myristate 13-acetate (PMA) and ionomycin, with PMA and anti-CD3/anti-CD28, with one or more antigens specifically recognized by one or more T cells of interest in the sample, or with extracts of cells or tissues. In some cases, a sample is divided into to a first sample whose T cells are stimulated and a second sample whose T cells are unstimulated, then the two samples are analyzed separately according to the method described herein.

In some cases, the second round of PCR in the present method of analyzing single T cells may involve splitting the amplification products encoding a TCR from the first round of PCR into two pools, and performing the second round of PCR in the first pool using a reverse primer that is specific to a first subtype of TCR, and in the second pool using a reverse primer that is specific to a second subtype of TCR. In such instances, the amplification product from the first pool and the second pool may include different T cell receptor chains (e.g., alpha and beta chains). For example, the first pool may amplify a TCR with an alpha chain and the second pool a TCR with a beta chain. As before, amplification products from the second round of PCR performed on amplification products originating from all or a subset of the separate locations containing a single T cell may be combined for sequencing.

In certain embodiments, the present method of analyzing single T cells is an efficient method of analyzing nucleic acids expressed in single T cells. The presence of a T cell receptor may be detected by the present method in 70% or more, e.g., 80% or more, 85% or more, 90% or more, 92% or more, or 94% or more, and in some cases 100% or less, e.g., 95% or less, or 94% or less of the single T cells sorted into the separate locations. In some instances, the presence of a T cell receptor may be detected by the present method in a range of 70 to 100%, e.g., a range of 80 to 98%, a range of 85 to 95%, including a range of 90 to 94% of the single T cells sorted into the separate locations. In some embodiments, presence of a T cell receptor alpha chain may be detected by the present method in 70% or more, e.g., 80% or more, 85% or more, or 90% or more, and in some cases 100% or less, e.g., 95% or less, or 90% or less of the single T cells sorted into the separate locations. In some instances, the presence of a T cell receptor alpha chain may be detected by the present method in a range of 70 to 100%, e.g., a range of 75 to 95%, a range of 80 to 92%, including a range of 85 to 90% of the single T cells sorted into the separate locations. In some embodiments, presence of a T cell receptor beta chain may be detected by the present method in 85% or more, e.g., 90% or more, or 94% or more, and in some cases 100% or less, e.g., 97% or less, or 94% or less of the single T cells sorted into the separate locations. In some instances, the presence of a T cell receptor beta chain may be detected by the present method in a range of 85 to 100%, e.g., a range of 98 to 98%, a range of 90 to 96%, including a range of 91 to 95% of the single T cells sorted into the separate locations.

In certain embodiments, the present method of analyzing single T cells is a sensitive method of analyzing nucleic acids expressed in single T cells. The present method may provide for detecting the presence of 50 molecules or less, e.g., 25 molecules or less, 20 molecules or less, 10 molecules or less, and down to 2 molecules of a target nucleic acid (e.g., mRNA for a T cell receptor) in a single T cell.

The technology described herein provides highly efficient TCR sequencing of single T cells and finds numerous applications in basic research and development. This methodology can be performed at reasonable cost by any standardly equipped laboratory with access to flow cytometry and deep sequencing. Sequencing TCRs of single T cells provides information about the ancestry of particular T cells. Furthermore, the sequences of nucleic acids amplified from T cells can be analyzed for splice variations, somatic mutations, or genetic polymorphisms. Of particular interest are genetic variations and mutations associated with immune disorders or cancer.

Additionally, knowledge of the sequences of TCRs from individual cells allows TCRs to be reconstituted for functional studies. For example, after analyzing a T cell as described herein and identifying a sequence encoding a TCRα polypeptide and a sequence encoding a TCRβ polypeptide from a single T cell, recombinant constructs expressing the TCRαβ heterodimer can be constructed. A host cell can be transformed with one or more recombinant polynucleotides encoding the TCR (e.g., separate monocistronic constructs expressing each polypeptide chain of the TCR heterodimer or a bicistronic construct expressing both the TCRα polypeptide and the TCR beta polypeptide). The TCR of the single cell can be produced by culturing the host cell under conditions suitable for the expression of the TCRα polypeptide and the TCRβ polypeptide and recovering the TCRαβ heterodimer from the host cell culture.

The reconstituted TCR can be used in screening to determine the target antigen bound by the TCR by contacting the TCR with potential target antigens displayed in complexes with major histocompatibility complex (MHC) and determining whether or not the target antigen binds to the TCR. The TCR can be screened for antigen binding in a high-throughput manner by providing a peptide library including a plurality of peptides displayed by major histocompatibility complex (MHC) molecules; and contacting the plurality of peptides with the TCR; and identifying at least one peptide-MHC complex that binds to the TCR. Any suitable antigen may find use in the present method. Exemplary antigens include, but are not limited to, antigenic molecules from infectious agents, auto-/self-antigens, tumor-/cancer-associated antigens, etc.

The present disclosure also provides kits for carrying out the method of the present disclosure. The above-described reagents, including the primers for amplification of target nucleic acid molecules encoding TCRs, and optionally other reagents for performing nucleic acid amplification (e.g., by RT-PCR) and/or sequencing can be provided in kits with suitable instructions and other necessary reagents for analyzing single T cells. The kit will normally contain in separate containers the primers and other reagents (e.g., polymerases, nucleoside triphosphates, and buffers). All primers within a set of primers may in some cases be provided in one container. In some cases, different subsets of primers within a set of primers may be provided in separate containers. Instructions (e.g., written, CD-ROM, DVD, flash drive, etc.) for carrying out the analysis of T cells usually will be included in the kit. The kit can also contain other packaged reagents and materials (i.e., wash buffers, cell lysis agents, reagents for extraction and purification of nucleic acids, and the like). Analysis of single T cells, as described herein, can be conducted using these kits.

Thus, the present disclosure provides kits that find use in performing the present method, as described above. In certain embodiments, the kit includes a first set of primers, which includes (a) a first set of primers, wherein the first set of primers comprises: (i) a first set of forward primers comprising the nucleotide sequences of SEQ ID NOs:1-40 and SEQ ID NOs:42-70 having a length ranging from 15 to 40 nucleotides, (ii) a first set of reverse primers comprising the nucleotide sequences of SEQ ID NOs:41 and 71 having a length ranging from 15 to 40 nucleotides; and (b) a second set of primers, wherein the second set of primers comprises: (i) a second set of forward primers comprising the nucleotide sequences of SEQ ID NOs:72-111 and SEQ ID NO:130-158 having a length ranging from 40 to 70 nucleotides, and (ii) a second set of reverse primers comprising the nucleotide sequences of SEQ ID NOs:112-129 and SEQ ID NOs:159-176 having a length ranging from 50 to 90 nucleotides; or (c) a first set of primers, wherein the first set of primers comprises: (i) a first set of forward primers comprising the nucleotide sequences of SEQ ID NOs:177-199 and SEQ ID NOs:201-217 having a length ranging from 15 to 40 nucleotides, (ii) a first set of reverse primers comprising the nucleotide sequences of SEQ ID NOs:200 and 218 having a length ranging from 15 to 40 nucleotides; and (d) a second set of primers, wherein the second set of primers comprises: (i) a second set of forward primers comprising the nucleotide sequences of SEQ ID NOs:219-244 and SEQ ID NO:263-289 having a length ranging from 40 to 70 nucleotides, and (ii) a second set of reverse primers comprising the nucleotide sequences of SEQ ID NOs:245-262 and SEQ ID NOs:290-307 having a length ranging from 50 to 90 nucleotides. In certain embodiments, the first set and second set of primers are included in separate containers. In other embodiments, the first set of forward primers, first set of reverse primers, second set of forward primers, and second set of reverse primers are each in separate containers.

The following non-limiting examples are provided to further illustrate the present invention.

### Example 1: Materials and Methods

The unbiased paired analysis of T-cell receptor (TCR) α- and β-chain usage at the single-cell level provides a valuable window for understanding the TCR repertoire and the nature of the immune response that would otherwise be difficult to obtain. Earlier technologies for TCR repertoire analysis were often limited to examining TCR complementarity-determining region 3 (CDR3) β expression or required in vitro cloning procedures that can artificially skew the TCR repertoire from the in vivo state. The protocol described here is a direct ex vivo, single-cell-based strategy for the clonotypic analysis of TCRαβ repertoires that uses multiplexed panels of CDR3α- and CDR3β-specific primers in a nested PCR to amplify transcripts from an individual, epitope-specific or naïve T cell by an next generation sequencing method.

Primers. External primers targeting human T cell receptor α (hTRA) and β (hTRB) genes for the first round of PCR amplification are provided in Tables 1 and 2, respectively.

TABLE 1

| hTRA gene(s) | External primer | |
|---|---|---|
| targeted by | sequence | SEQ ID |
| primer | (5′->3′) | NO: |

Forward

| | | |
|---|---|---|
| huTRAV1ext | AACTGCACGTACCAGACATC | |
| huTRAV2ext | GATGTGCACCAAGACTCC | 2 |
| huTRAV3ext | AAGATCAGGTCAACGTTGC | 3 |
| huTRAV4ext | CTCCATGGACTCATATGAAGG | 4 |
| huTRAV5ext | CTTTTCCTGAGTGTCCGAG | 5 |
| huTRAV6ext | CACCCTGACCTGCAACTATAC | 6 |
| huTRAV7ext | GCAAAATACAGGGATGGG | 7 |
| huTRAV8-1ext | CTCACTGGAGTTGGGATG | 8 |
| huTRAV8-3ext | CACTGTCTCTGAAGGAGCC | 9 |
| huTRAV8-2,4ext | GCCACCCTGGTTAAAGG | 10 |
| huTRAV8-6ext | GAGCTGAGGTGCAACTACTC | 11 |
| huTRAV8-7ext2 | CTAACAGAGGCCACCCAG | 12 |
| huTRAV9-1_2ext | TGGTATGTCCAATATCCTGG | 13 |
| huTRAV10ext | CAAGTGGAGCAGAGTCCTC | 14 |
| huTRAV12-1_3ext | CARTGTTCCAGAGGGAGC | 15 |
| huTRAV13-1ext | CATCCTTCAACCCTGAGTG | 16 |
| huTRAV13-2ext | CAGCGCCTCAGACTACTTC | 17 |
| huTRAV14ext | AAGATAACTCAAACCCAACCAG | 18 |
| huTRAV16ext | AGTGGAGCTGAAGTGCAAC | 19 |
| huTRAV17ext | GGAGAAGAGGATCCTCAGG | 20 |
| huTRAV18ext3 | TCCAGTATCTAAACAAAGAGCC | 21 |
| huTRAV19ext | AGGTAACTCAAGCGCAGAC | 22 |
| huTRAV20ext | CACAGTCAGCGGTTTAAGAG | 23 |
| huTRAV21ext | TTCCTGCAGCTCTGAGTG | 24 |
| huTRAV22ext | GTCCTCCAGACCTGATTCTC | 25 |
| huTRAV23ext | TGCTTATGAGAACACTGCG | 26 |
| huTRAV24ext | CTCAGTCACTGCATGTTCAG | 27 |
| huTRAV25ext | GGACTTCACCACGTACTGC | 28 |
| huTRAV26-1ext | GCAAACCTGCCTTGTAATC | 29 |
| huTRAV26-2ext | AGCCAAATTCAATGGAGAG | 30 |
| huTRAV27ext | TCAGTTTCTAAGCATCCAAGAG | 31 |
| huTRAV29ext | GCAAGTTAAGCAAAATTCACC | 32 |
| huTRAV30ext | CAACAACCAGTGCAGAGTC | 33 |
| huTRAV34ext | AGAACTGGAGCAGAGTCCTC | 34 |
| huTRAV35ext | GGTCAACAGCTGAATCAGAG | 35 |
| huTRAV36ext | GAAGACAAGGTGGTACAAAGC | 36 |
| huTRAV38ext | GCACATATGACACCAGTGAG | 37 |
| huTRAV39ext | CTGTTCCTGAGCATGCAG. | 38 |
| huTRAV40ext | GCATCTGTGACTATGAACTGC | 39 |
| huTRAV41ext | AATGAAGTGGAGCAGAGTCC | 40 |

Reverse

| | | |
|---|---|---|
| hTRAC | GACCAGCTTGACATCACAG | 41 |

Primers targeting hTRAV are sense.
Primers targeting hTRAC genes are antisense.
hTRAV, human T cell receptor Vα; hTRAC, human T cell receptor Cα.

TABLE 2

| hTRB gene(s) | External primer | SEQ |
|---|---|---|
| targeted | sequence | ID |
| by primer | (5′->3′) | NO: |

Forward

| | | |
|---|---|---|
| huTRBV2ext | TCGATGATCAATTCTCAGTTG | 42 |
| huTRBV3ext | CAAAATACCTGGTCACACAG | 43 |
| huTRBV4ext | TCGCTTCTCACCTGAATG | 44 |
| huTRBV5-1_4ext | GATTCTCAGGKCKCCAGTTC | 45 |
| huTRBV5-5_8ext | GTACCAACAGGYCCTGGGT | 46 |
| huTRBV6-1_3, | ACTCAGACCCCAAAATTCC | 47 |
| 5_9ext | | |
| huTRBV6-4ext | ACTGGCAAAGGAGAAGTCC | 48 |
| huTRBV7-1_3ext | TRTGATCCAATTTCAGGTCA | 49 |
| huTRBV7-4_9ext_ | CGSWTCTYTGCAGARAGGC | 50 |
| new | | |
| huTRBV9ext | GATCACAGCAACTGGACAG | 51 |
| huTRBV10-1ext | CAGAGCCCAAGACACAAG | 52 |
| huTRBV10-2ext | ACCTTGATGTGTCACCAGAC | 53 |
| huTRBV10-3ext | CAGAGCCCAAGACACAAG | 54 |
| huTRBV11ext | CGATTTTCTGCAGAGACGC | 55 |
| huTRBV12ext | ARGTGACAGARATGGGACAA | 56 |
| huTRBV13ext | AGCGATAAAGGAAGCATCC | 57 |
| huTRBV14ext | CCAACAATCGATTCTTAGCTG | 58 |
| huTRBV15ext | AGTGACCCTGAGTTGTTCTC | 59 |
| huTRBV16ext | GTCTTTGATGAAACAGGTATGC | 60 |
| huTRBV17ext | CAGACCCCCAGACACAAG | 61 |
| huTRBV18ext | CATAGATGAGTCAGGAATGCC | 62 |
| huTRBV19ext | AGTTGTGAACAGAATTTGAACC | 63 |
| huTRBV20ext | AAGTTTCTCATCAACCATGC | 64 |
| huTRBV23ext | GCGATTCTCATCTCAATGC | 65 |
| huTRBV24ext | CCTACGGTTGATCTATTACTCC | 66 |
| huTRBV25ext | ACTACACCTCATCCACTATTCC | 67 |
| huTRBV27, | TGGTATCGACAAGACCCAG | 68 |
| 28ext | | |
| huTRBV29ext | TTCTGGTACCGTCAGCAAC | 69 |
| huTRBV30ext | TCCAGCTGCTCTTCTACTCC | 70 |

Reverse

| | | |
|---|---|---|
| hTRBC | TAGAACTGGACTTGACAGCG | 71 |

Primers targeting hTRBV genes are sense.
Primers targeting hTRBC genes are antisense.
hTRBV, human T cell receptor Vβ; hTRBC, human T cell receptor Cβ.

Internal primers for nested PCR amplification of hTRA and hTRB amplicons in the second round of PCR amplification are provided in Tables 3 and 4, respectively.

TABLE 3

| hTRA | | |
|---|---|---|
| gene(s) | | |
| targeted | Internal primer | SEQ |
| by | sequence | ID |
| primer | (5′->3′) | NO: |

Forward

| | | |
|---|---|---|
| hTRAV1 | tcgtcggcagcgtcagatgtgtataa | 72 |
| | gagacagGCACCCACATTTCTKTCTTAC | |
| hTRAV2 | tcgtcggcagcgtcagatgtgtataa | 73 |
| | gagacagCCTTCTCAGCAGGGACG | |
| hTRAV3 | tcgtcggcagcgtcagatgtgtataa | 74 |
| | gagacagACCGAGGCCTCCAGTTC | |
| hTRAV4 | tcgtcggcagcgtcagatgtgtataa | 75 |
| | gagacagAGTGGCCTCCCTGTTTATC | |
| hTRAV5 | tcgtcggcagcgtcagatgtgtataa | 76 |
| | gagacagCCAAAGACTCACTGTTCTATTG | |
| hTRAV6 | tcgtcggcagcgtcagatgtgtataa | 77 |
| | gagacagACAAGATCCAGGAAGAGGC | |
| hTRAV7 | tcgtcggcagcgtcagatgtgtataa | 78 |
| | gagacagTATGAGAAGCAGAAAGGAAGAC | |
| hTRAV8-1 | tcgtcggcagcgtcagatgtgtataa | 79 |
| | gagacagGTCAACACCTTCAGCTTCTC | |
| hTRAV8-2, | tcgtcggcagcgtcagatgtgtataa | 80 |
| 8-4 | gagacagAGAGTGAAACCTCCTTCCAC | |
| hTRAV8-3 | tcgtcggcagcgtcagatgtgtataa | 81 |
| | gagacagTTTGAGGCTGAATTTAAGAGG | |
| hTRAV8-6 | tcgtcggcagcgtcagatgtgtataa | 82 |
| | gagacagAACCAAGGACTCCAGCTTC | |
| hTRAV8-7 | tcgtcggcagcgtcagatgtgtataa | 83 |
| | gagacagATCAGAGGTTTTGAGGCTG | |
| hTRAV9-1, | tcgtcggcagcgtcagatgtgtataa | 84 |
| 9-2 | gagacagGAAACCACTTCTTTCCACTTG | |
| hTRAV10 | tcgtcggcagcgtcagatgtgtataa | 85 |
| | gagacagGAAGATATACAGCAACTCTGG | |
| hTRAV12-1, | tcgtcggcagcgtcagatgtgtataa | 86 |
| 12-2, 12-3 | gagacagAAGATGGAAGGTTTACAGCAC | |
| hTRAV13-1 | tcgtcggcagcgtcagatgtgtataa | 87 |
| | gagacagCCAACGAATTGCTGTTACATTG | |
| hTRAV13-2 | tcgtcggcagcgtcagatgtgtataa | 88 |
| | gagacagCAGTGAAACATCTCTCTCTGC | |
| hTRAV14 | tcgtcggcagcgtcagatgtgtataa | 89 |
| | gagacagCAACAGAAGGTCGCTACTC | |
| hTRAV16 | tcgtcggcagcgtcagatgtgtataa | 90 |
| | gagacagGTCCAGTACTCCAGACAACG | |
| hTRAV17 | tcgtcggcagcgtcagatgtgtataa | 91 |
| | gagacagCAGTGGAAGATTAAGAGTCAC | |
| hTRAV18 | tcgtcggcagcgtcagatgtgtataa | 92 |
| | gagacagTGACAGTTCCTTCCACCTG | |
| hTRAV19 | tcgtcggcagcgtcagatgtgtataa | 93 |
| | gagacagGTGGTCGGTATTCTTGGAAC | |
| hTRAV20 | tcgtcggcagcgtcagatgtgtataa | 94 |
| | gagacagCTCTTCACCCTGTATTCAGC | |
| hTRAV21 | tcgtcggcagcgtcagatgtgtataa | 95 |
| | gagacagGTGGAAGACTTAATGCCTCG | |
| hTRAV22 | tcgtcggcagcgtcagatgtgtataa | 96 |
| | gagacagGCCACGACTGTCGCTAC | |
| hTRAV23 | tcgtcggcagcgtcagatgtgtataa | 97 |
| | gagacagTGCATTATTGATAGCCATACG | |
| hTRAV24 | tcgtcggcagcgtcagatgtgtataa | 98 |
| | gagacagGGACGAATAAGTGCCACTC | |
| hTRAV25 | tcgtcggcagcgtcagatgtgtataa | 99 |
| | gagacagTGACATTTCAGTTTGGAGAAGC | |
| hTRAV26-1 | tcgtcggcagcgtcagatgtgtataa | 100 |
| | gagacagCGACAGATTCACTCCCAG | |
| hTRAV26-2 | tcgtcggcagcgtcagatgtgtataa | 101 |
| | gagacagGCCTCTCTGGCAATCGC | |
| hTRAV27 | tcgtcggcagcgtcagatgtgtataa | 102 |
| | gagacagGAAGCTGAAGAGACTAACCTT | |
| hTRAV29 | tcgtcggcagcgtcagatgtgtataa | 103 |
| | gagacagCTGCTGAAGGTCCTACATTC | |
| hTRAV30 | tcgtcggcagcgtcagatgtgtataa | 104 |
| | gagacagAGAAGCATGGTGAAGCAC | |
| hTRAV34 | tcgtcggcagcgtcagatgtgtataa | 105 |
| | gagacagTCATGAAAAGATAACTGCCAAG | |
| hTRAV35 | tcgtcggcagcgtcagatgtgtataa | 106 |
| | gagacagTGGAAGACTGACTGCTCAG | |
| hTRAV36 | tcgtcggcagcgtcagatgtgtataa | 107 |
| | gagacagGTCAGGAAGACTAAGTAGC | |
| hTRAV38 | tcgtcggcagcgtcagatgtgtataa | 108 |
| | gagacagCAGCAGGCAGATGATTCTC | |
| hTRAV39 | tcgtcggcagcgtcagatgtgtataa | 109 |
| | gagacagGTGTTGCTATCAAATGGAGC | |
| hTRAV90 | tcgtcggcagcgtcagatgtgtataa | 110 |
| | gagacagGGAGGCGGAAATATTAAAGAC | |
| hTRAV41 | tcgtcggcagcgtcagatgtgtataa | 111 |
| | gagacagTTGTTTATGCTGAGCTCAGG | |

Reverse

| | | |
|---|---|---|
| A-hTRAC | gtctcgtgggctcggagatgtgtataa | 112 |
| | gagacag [symbol] TGTTRACCACTRAC | |
| | TGGTRACATTTRACTAGTRACAGTTRACCTC | |
| B-hTRAC | gtctcgtgggctcggagatgtgtataa | 113 |
| | gagacag [symbol] TGTTRACCACTRAC | |
| | TGGTRACATTTRACTAGTRACAGTTRACCTC | |
| C-hTRAC | gtctcgtgggctcggagatgtgtataa | 114 |
| | gagacag [symbol] TGTTRACCACTRAC | |
| | TGGTRACATTTRACTAGTRACAGTTRACCTC | |
| D-hTRAC | gtctcgtgggctcggagatgtgtataa | 115 |
| | gagacag [symbol] TGTTRACCACTRAC | |
| | TGGTRACATTTRACTAGTRACAGTTRACCTC | |
| E-hTRAC | gtctcgtgggctcggagatgtgtataa | 116 |
| | gagacag [symbol] TGTTRACCACTRAC | |
| | TGGTRACATTTRACTAGTRACAGTTRACCTC | |
| F-hTRAC | gtctcgtgggctcggagatgtgtataa | 117 |
| | gagacag [symbol] TGTTRACCACTRAC | |
| | TGGTRACATTTRACTAGTRACAGTTRACCTC | |
| G-hTRAC | gtctcgtgggctcggagatgtgtataa | 118 |
| | gagacag [symbol] TGTTRACCACTRAC | |
| | TGGTRACATTTRACTAGTRACAGTTRACCTC | |
| H-hTRAC | gtctcgtgggctcggagatgtgtataa | 119 |
| | gagacag [symbol] TGTTRACCACTRAC | |
| | TGGTRACATTTRACTAGTRACAGTTRACCTC | |
| I-hTRAC | gtctcgtgggctcggagatgtgtataa | 120 |
| | gagacag [symbol] TGTTRACCACTRAC | |
| | TGGTRACATTTRACTAGTRACAGTTRACCTC | |
| J-hTRAC | gtctcgtgggctcggagatgtgtataa | 121 |
| | gagacag [symbol] TGTTRACCACTRAC | |
| | TGGTRACATTTRACTAGTRACAGTTRACCTC | |
| K-hTRAC | gtctcgtgggctcggagatgtgtataa | 122 |
| | gagacag [symbol] TGTTRACCACTRAC | |
| | TGGTRACATTTRACTAGTRACAGTTRACCTC | |
| L-hTRAC | gtctcgtgggctcggagatgtgtataa | 123 |
| | gagacag [symbol] TGTTRACCACTRAC | |
| | TGGTRACATTTRACTAGTRACAGTTRACCTC | |
| M-hTRAC | gtctcgtgggctcggagatgtgtataa | 124 |
| | gagacag [symbol] TGTTRACCACTRAC | |
| | TGGTRACATTTRACTAGTRACAGTTRACCTC | |
| N-hTRAC | gtctcgtgggctcggagatgtgtataa | 125 |
| | gagacag [symbol] TGTTRACCACTRAC | |
| | TGGTRACATTTRACTAGTRACAGTTRACCTC | |
| O-hTRAC | gtctcgtgggctcggagatgtgtataa | 126 |
| | gagacag [symbol] TGTTRACCACTRAC | |
| | TGGTRACATTTRACTAGTRACAGTTRACCTC | |
| P-hTRAC | gtctcgtgggctcggagatgtgtataa | 127 |
| | gagacagggacggTGTTRACCACTRAC | |
| | TGGTRACATTTRACTAGTRACAGTTRACCTC | |
| Q-hTRAC | gtctcgtgggctcggagatgtgtataa | 128 |
| | gagacag [symbol] TGTTRACCACTRAC | |
| | TGGTRACATTTRACTAGTRACAGTTRACCTC | |
| R-hTRAC | gtctcgtgggctcggagatgtgtataa | 129 |
| | gagacag [symbol] TGTTRACCACTRAC | |
| | TGGTRACATTTRACTAGTRACAGTTRACCTC | |

Primers targeting hTRAV are sense.
Primers targeting hTRAC genes are antisense.
hTRAV, human T cell receptor Vα; hTRAC, human T cell receptor Cα.
Lower case letters, adapter sequences (ILLUMINA®).
Bold and underlined sequences, barcode sequences (ILLUMINA®).

TABLE 4

| hTRB | | |
|---|---|---|
| gene(s) | | SEQ |
| targeted | Internal primer sequence | ID |
| by primer | (5'->3') | NO: |

Forward

| | | |
|---|---|---|
| hTRBV2 | tcgtcggcagcgtcagatgtgtataa | 130 |
| | gagacagTTCACTCTGAAGATCCGGTC | |
| hTRBV3 | tcgtcggcagcgtcagatgtgtataa | 131 |
| | gagacagAATCTTCACATCAATTCCCTG | |
| hTRBV4 | tcgtcggcagcgtcagatgtgtataa | 132 |
| | gagacagCCTGCAGCCAGAAGACTC | |
| hTRBV5-1, | tcgtcggcagcgtcagatgtgtataa | 133 |
| 5-2, 5-3, | gagacagCTTGGAGCTGGRSGACTC | |
| 5-4 | | |
| hTRBV5-5, | TCGTCGGCAGCGTCAGATGTGTATAA | 134 |
| 5-6, 5-7, | gagacagTCTGAGCTGAATGTGAACG | |
| 5-8 | | |
| hTRBV6-1, | tcgtcggcagcgtcagatgtgtataa | 135 |
| 6-2, 6-3, | gagacagATGGCTACAAYGTMTCYAG | |
| 6-5, 6-6, | | |
| 6-7, 6-8, | | |
| 6-9 | | |
| hTRBV6-4 | tcgtcggcagcgtcagatgtgtataa | 136 |
| | gagacagTGGTTATAGTGTCTCCAGAGC | |
| hTRBV7-1, | tcgtcggcagcgtcagatgtgtataa | 137 |
| 7-2, 7-3 | gagacagTGYACTCTGAMGWTCCAGCG | |
| hTRBV7-4, | tcgtcggcagcgtcagatgtgtataa | 138 |
| 7-5, 7-6, | gagacagTGRMGATYCAGCGCACA | |
| 7-7, | | |
| 7-8, 7-9 | | |
| hTRBV9 | tcgtcggcagcgtcagatgtgtataa | 139 |
| | gagacagGAAACATTCTTGAACGATTCTC | |
| hTRBV10-1 | tcgtcggcagcgtcagatgtgtataa | 140 |
| | gagacagTGGTATCGACAAGACCTGG | |
| hTRBV10-2 | tcgtcggcagcgtcagatgtgtataa | 141 |
| | gagacagTGGTATCGACAAGACCTGG | |
| hTRBV10-3 | tcgtcggcagcgtcagatgtgtataa | 142 |
| | gagacagGGAACACCAGTGACTCTGAG | |
| hTRBV11 | tcgtcggcagcgtcagatgtgtataa | 143 |
| | gagacagGACTCCACTCTCAAGATCCA | |
| hTRBV12 | tcgtcggcagcgtcagatgtgtataa | 144 |
| | gagacagCYACTCTGARGATCCAGCC | |
| hTRBV13 | tcgtcggcagcgtcagatgtgtataa | 145 |
| | gagacagCATTCTGAACTGAACATGAGC | |
| hTRBV14 | tcgtcggcagcgtcagatgtgtataa | 146 |
| | gagacagATTCTACTCTGAAGGTGCAGC | |
| hTRBV15 | tcgtcggcagcgtcagatgtgtataa | 147 |
| | gagacagATAACTTCCAATCCAGGAGG | |
| hTRBV16 | tcgtcggcagcgtcagatgtgtataa | 148 |
| | gagacagCTGTAGCCTTGAGATCCAGG | |
| hTRBV17 | tcgtcggcagcgtcagatgtgtataa | 149 |
| | gagacagGATGCCCAAGGAACGATTC | |
| hTRBV18 | tcgtcggcagcgtcagatgtgtataa | 150 |
| | gagacagCGATTTTCTGCTGAATTTCC | |
| hTRBV19 | tcgtcggcagcgtcagatgtgtataa | 151 |
| | gagacagTTCCTCTCACTGTGACATCG | |
| hTRBV20 | tcgtcggcagcgtcagatgtgtataa | 152 |
| | gagacagACTCTGACAGTGACCAGTGC | |
| hTRBV23 | tcgtcggcagcgtcagatgtgtataa | 153 |
| | gagacagGCAATCCTGTCCTCAGAAC | |
| hTRBV24 | tcgtcggcagcgtcagatgtgtataa | 154 |
| | gagacagGATGGATACAGTGITTCTCGA | |
| hTRBV25 | tcgtcggcagcgtcagatgtgtataa | 155 |
| | gagacagCAGAGAAGGGAGATCTTTCC | |
| hTRBV27, | tcgtcggcagcgtcagatgtgtataa | 156 |
| 28 | gagacagTTCYCCCTGATYCTGGAGTC | |
| hTRBV29 | tcgtcggcagcgtcagatgtgtataa | 157 |
| | gagacagTCTGACTGTGAGCAACATGAG | |
| hTRBV30 | tcgtcggcagcgtcagatgtgtataa | 158 |
| | gagacagAGAATCTCTCAGCCTCCAGAC | |

Reverse

| | | |
|---|---|---|
| A-hTRBC | gtctcgtgggctcggagatgtgtataa | 159 |
| | gagacag [symbol] TTCTGATGGCTCAAACACAG | |
| B-hTRBC | gtctcgtgggctcggagatgtgtataa | 160 |
| | gagacag [symbol] TTCTGATGGCTCAAACACAG | |
| C-hTRBC | gtctcgtgggctcggagatgtgtataa | 161 |
| | gagacag [symbol] TTCTGATGGCTCAAACACAG | |
| D-hTRBC | gtctcgtgggctcggagatgtgtataa | 162 |
| | gagacag [symbol] TTCTGATGGCTCAAACACAG | |
| E-hTRBC | gtctcgtgggctcggagatgtgtataa | 163 |
| | gagacag [symbol] TTCTGATGGCTCAAACACAG | |
| F-hTRBC | gtctcgtgggctcggagatgtgtataa | 164 |
| | gagacag [symbol] TTCTGATGGCTCAAACACAG | |
| G-hTRBC | gtctcgtgggctcggagatgtgtataa | 165 |
| | gagacag [symbol] TTCTGATGGCTCAAACACAG | |
| H-hTRBC | gtctcgtgggctcggagatgtgtataa | 166 |
| | gagacag [symbol] TTCTGATGGCTCAAACACAG | |
| I-hTRBC | gtctcgtgggctcggagatgtgtataa | 167 |
| | gagacag [symbol] TTCTGATGGCTCAAACACAG | |
| J-hTRBC | gtctcgtgggctcggagatgtgtataa | 160 |
| | gagacag [symbol] TTCTGATGGCTCAAACACAG | |
| K-hTRBC | gtctcgtgggctcggagatgtgtataa | 169 |
| | gagacag [symbol] TTCTGATGGCTCAAACACAG | |
| L-hTRBC | gtctcgtgggctcggagatgtgtataa | 170 |
| | gagacag [symbol] TTCTGATGGCTCAAACACAG | |
| M-hTRBC | gtctcgtgggctcggagatgtgtataa | 171 |
| | gagacag [symbol] TTCTGATGGCTCAAACACAG | |
| N-hTRBC | gtctcgtgggctcggagatgtgtataa | 172 |
| | gagacag [symbol] TTCTGATGGCTCAAACACAG | |
| O-hTRBC | gtctcgtgggctcggagatgtgtataa | 173 |
| | gagacag [symbol] TTCTGATGGCTCAAACACAG | |
| P-hTRBC | gtctcgtgggctcggagatgtgtataa | 174 |
| | gagacag [symbol] TTCTGATGGCTCAAACACAG | |
| Q-hTRBC | gtctcgtgggctcggagatgtgtataa | 175 |
| | gagacag [symbol] TTCTGATGGCTCAAACACAG | |
| R-hTRBC | gtctcgtgggctcggagatgtgtataa | 176 |
| | gagacag [symbol] TTCTGATGGCTCAAACACAG | |

Primers targeting hTRBV genes are sense.
Primers targeting hTRBC genes are antisense.
hTRBV, human T cell receptor Vβ; hTRBC, human T cell receptor cβ.
Lower case letters, adapter sequences (ILLUMIN®).
Bold and underlined sequences, barcode sequences (ILLUMIN®).

External primers targeting mouse T cell receptor α (mTRA) and β (mTRB) genes for the first round of FOR amplification are provided in Tables 5 and 6, respectively.

TABLE 5

| mTRA gene(s) | External primer | SEQ |
|---|---|---|
| targeted | sequence | ID |
| by primer | (5′->3′) | NO: |

Forward

| | | |
|---|---|---|
| mTRAV1Ext | GGTTATCCTGGTACCAGCA | 177 |
| mTRAV2Ext | CATCTACTGGTACCGACAGG | 178 |
| mTRAV3Ext | GGCGAGCAGGTGGAG | 179 |
| mTRAV4Ext | TCTGSTCTGAGATGCAATTTT | 180 |
| mTRAV5-1/5-4 | GGCTACTTCCCTTGGTATAAGCAAGA | 181 |
| (D)Ext | | |
| mTRAV6-1/6- | CAGATGCAAGGTCAAGTGAC | 182 |
| 2Ext | | |
| mTRAV6-3/6-4 | AAGGTCCACAGCTCCTTC | 183 |
| (D)Ext | | |
| mTRAV6-5/6-7 | GTTCTGGTATGTGCAGTATCC | 184 |
| (D)Ext | | |
| mTRAV6-6Ext | AGATTCCGTGACTCAAACAG | 185 |
| mTRAV7Ext | AGAAGGTRCAGCAGAGCCCAGAATC | 186 |
| mTRAV8Ext | GAGCRTCCASGAGGGTG | 187 |
| mTRAV9Ext | CCAGTGGTTCAAGGAGTG | 188 |
| mTRAV10/10a | AGAGAAGGTCGAGCAACAC | 189 |
| (D)Ext | | |
| mTRAV11Ext | AAGACCCAAGTGGAGCAG | 190 |
| mTRAV12Ext | TGACCCAGACAGAAGGC | 191 |
| mTRAV13Ext | TCCTTGGTTCTGCAGG | 192 |
| mTRAV14Ext | GCAGCAGGTGAGACAAAG | 193 |
| mTRAV15Ext | CASCTTYTTAGTGGAGAGATGG | 194 |
| mTRAV16Ext | GTACAAGCAAACAGCAAGTG | 195 |
| mTRAV17Ext | CAGTCCGTGGACCAGC | 196 |
| mTRAV18Ext | AACGGCTGGAGCAGAG | 197 |
| mTRAV19Ext | GCAAGTTAAACAAAGCTCTCC | 198 |
| mTRAV21Ext | GTGCACTTGCCTTGTAGC | 199 |

Reverse

| | | |
|---|---|---|
| mTRAC | GGCATCACAGGGAACG | 200 |

Primers targeting mTRAV are sense.
Primers targeting mTRAC genes are antisense.
mTRAV, mouse T cell receptor Vα; mTRAC, mouse T cell receptor Cα.

TABLE 6

| mTRB gene(s) | External primer | SEQ |
|---|---|---|
| targeted by | sequence | ID |
| primer | (5′->3′) | NO: |

Forward

| | | |
|---|---|---|
| mTRBV1Ext | TACCACGTGGTCAAGCTG | 201 |
| mTRBV2Ext | CAGTATCTAGGCCACAATGC | 202 |
| mTRBV3Ext | CCCAAAGTCTTACAGATCCC | 203 |
| mTRBV4Ext | GACGGCTGTTTTCCAGAC | 204 |
| mTRBV5Ext | GGTATAAACAGAGCGCTGAG | 205 |
| mTRBV12Ext | GGGGTTGTCCAGTCTCC | 206 |
| mTRBV13Ext | GCTGCAGTCACCCAAAG | 207 |
| mTRBV14Ext | GCAGTCCTACAGGAAGGG | 208 |
| mTRBV15Ext | GAGTTACCCAGACACCCAG | 209 |
| mTRBV16Ext | CCTAGGCACAAGGTGACAG | 210 |
| mTRBV17Ext | GAAGCCAAACCAAGCAC | 211 |
| mTRBV19Ext | GATTGGTCAGGAAGGGC | 212 |
| mTRBV20Ext | GGATGGAGTGTCAAGCTG | 213 |
| mTRBV23Ext | CTGCAGTTACACAGAAGCC | 214 |
| mTRBV24Ext | CAGACTCCACGATACCTGG | 215 |
| mTRBV26Ext | GGTGAAAGGGCAAGGAC | 216 |
| mTRBV29Ext | GCTGGAATGTGGACAGG | 217 |

Reverse

| | | |
|---|---|---|
| mTRBC | CCAGAAGGTAGCAGAGACCC | 218 |

Primers targeting mTRBV genes are sense.
Primers targeting mTRBC genes are antisense.
mTRBV, mouse T cell receptor Vβ; mTRBC, mouse T cell receptor Cβ.

Internal primers for nested PCR amplification of mTRA and mTRB amplicons in the second round of PCR amplification are provided in Tables 7 and 8, respectively.

TABLE 7

| mTRA | | |
|---|---|---|
| gene(s) | | SEQ |
| targeted | Internal primer sequence | ID |
| by primer | (5′->3′) | NO: |

Forward

| | | |
|---|---|---|
| mTRAV12 | tcgtcggcagcgtcagatgtgtataagagacag | 219 |
| | CGCCACTCTCCATAAG | |
| mTRAV6 | tcgtcggcagcgtcagatgtgtataagagacag | 220 |
| | CAGAGGBTTTGAAGC | |
| mTRAV5N- | tcgtcggcagcgtcagatgtgtataagagacag | 221 |
| 3.9 | CTCCTCAAGTACTATTC | |
| mTRAV17 | tcgtcggcagcgtcagatgtgtataagagacag | 222 |
| | GGCCAGAGCCTCCAG | |
| mTRAV1 | tcgtcggcagcgtcagatgtgtataagagacag | 223 |
| | GAAGGCCAMGCCCC | |
| mTRAV2 | tcgtcggcagcgtcagatgtgtataagagacag | 224 |
| | CACCAGGGACCACAG | |
| mTRAV21/ | tcgtcggcagcgtcagatgtgtataagagacag | 225 |
| DV12 | CTCTTCAGGGTCCAGA | |
| mTRAV15 | tcgtcggcagcgtcagatgtgtataagagacag | 226 |
| | TAGTGGAGAGATGGTTTT | |
| mTRAV16 | tcgtcggcagcgtcagatgtgtataagagacag | 227 |
| | GCAAGTGGGRAAATAGTT | |
| mTRAV10 | tcgtcggcagcgtcagatgtgtataagagacag | 228 |
| | TGGAAAGRGTCTCCAC | |
| mTRAV20 | tcgtcggcagcgtcagatgtgtataagagacag | 229 |
| | AACAGAAAGTTTTCACGC | |
| mTRAV3 | tcgtcggcagcgtcagatgtgtataagagacag | 230 |
| | CTYCAGTTSCTTATG | |
| mTRAV5 | tcgtcggcagcgtcagatgtgtataagagacag | 231 |
| | GTTSMTATGGAAAGA | |
| mTRAV10 | tcgtcggcagcgtcagatgtgtataagagacag | 232 |
| | GYCCYGCWCTYCTGATA | |
| mTRAV14 | tcgtcggcagcgtcagatgtgtataagagacag | 233 |
| | CAACAGCCSCACACTC | |
| mTRAV19 | tcgtcggcagcgtcagatgtgtataagagacag | 234 |
| | CTGGRRAARSCCCC | |
| mTRAV7 | tcgtcggcagcgtcagatgtgtataagagacag | 235 |
| | TGSGAAAGGCCTTGAG | |
| mTRAV7D- | tcgtcggcagcgtcagatgtgtataagagacag | 236 |
| 5 | CAGGCAAAGGTCTTGTG | |
| mTRAV11 | tcgtcggcagcgtcagatgtgtataagagacag | 237 |
| | CAGGCRRAGGCCCKG | |
| mTRAV8 | tcgtcggcagcgtcagatgtgtataagagacag | 238 |
| | CCAGGGAAGGTCCTG | |
| mTRAV18 | tcgtcggcagcgtcagatgtgtataagagacag | 239 |
| | TGGGGGAMGYCTCATC | |
| mTRAV13N- | tcgtcggcagcgtcagatgtgtataagagacag | 240 |
| 1.13N-5 | AAGHCTCGTCAGCCTG | |
| mTRAVl3 | tcgtcggcagcgtcagatgtgtataagagacag | 241 |
| | AGGAACAAAGGAGAA | |
| mTRAV4 | tcgtcggcagcgtcagatgtgtataagagacag | 242 |
| | GAAGATCAGTAGAAGATT | |
| mTRAV22 | tcgtcggcagcgtcagatgtgtataagagacag | 243 |
| | GAAGAGAGATAGAAGATT | |
| mTRAV23 | tcgtcggcagcgtcagatgtgtataagagacag | 244 |
| | CGCCACTCTCCATAAG | |

Reverse

| | | |
|---|---|---|
| A-mTRAC | gtctcgtgggctcggagatgtgtataagagacag | 245 |
| | cgtgatCTGTCCTGAGACCGAG | |
| B-mTRAC | gtctcgtgggctcggagatgtgtataagagacag | 246 |
| | acatcgCTGTCCTGAGACCGAG | |
| C-mTRAC | gtctcgtgggctcggagatgtgtataagagacag | 247 |
| | gcctaaCTGTCCTGAGACCGAG | |
| D-mTRAC | gtctcgtgggctcggagatgtgtataagagacag | 248 |
| | tggtcaCTGTCCTGAGACCGAG | |
| E-mTRAC | gtctcgtgggctcggagatgtgtataagagacag | 249 |
| | attggcCTGTCCTGAGACCGAG | |
| F-mTRAC | gtctcgtgggctcggagatgtgtataagagacag | 250 |
| | gatctgCTGTCCTGAGACCGAG | |
| G-mTRAC | gtctcgtgggctcggagatgtgtataagagacag | 251 |
| | tcaagtCTGTCCTGAGACCGAG | |
| H-mTRAC | gtctcgtgggctcggagatgtgtataagagacag | 252 |
| | ctgatcCTGTCCTGAGACCGAG | |
| I-mTRAC | gtctcgtgggctcggagatgtgtataagagacag | 253 |
| | aagctaCTGTCCTGAGACCGAG | |
| J-mTRAC | gtctcgtgggctcggagatgtgtataagagacag | 254 |
| | gtagccCTGTCCTGAGACCGAG | |
| K-mTRAC | gtctcgtgggctcggagatgtgtataagagacag | 255 |
| | tacaagCTGTCCTGAGACCGAG | |
| L-mTRAC | gtctcgtgggctcggagatgtgtataagagacag | 256 |
| | cactgtCTGTCCTGAGACCGAG | |
| M-mTRAC | gtctcgtgggctcggagatgtgtataagagacag | 257 |
| | ttgactCTGTCCTGAGACCGAG | |
| N-mTRAC | gtctcgtgggctcggagatgtgtataagagacag | 258 |
| | ggaactCTGTCCTGAGACCGAG | |
| O-mTRAC | gtctcgtgggctcggagatgtgtataagagacag | 259 |
| | tgacatCTGTCCTGAGACCGAG | |
| P-mTRAC | gtctcgtgggctcggagatgtgtataagagacag | 260 |
| | ggacggCTGTCCTGAGACCGAG | |
| Q-mTRAC | gtctcgtgggctcggagatgtgtataagagacag | 261 |
| | ctctacCTGTCCTGAGACCGAG | |
| R-mTRAC | gtctcgtgggctcggagatgtgtataagagacag | 262 |
| | gcggacCTGTCCTGAGACCGAG | |

Primers targeting mTRAV are sense.
Primers targeting mTRAC genes are antisense.
mTRAV, mouse T cell receptor Vα; mTRAC, mouse T cell receptor Cα.
Lower case letters, adapter sequences (ILLUMINA®).
Bold and underlined sequences, barcode sequences (ILLUMINA®).

TABLE 8
mTRB

| gene(s) | | SEQ |
|---|---|---|
| targeted | Internal primer sequence | ID |
| by primer | (5′->3′) | NO: |

Forward

| | | |
|---|---|---|
| mTRBV | tcgtcggcagcgtcagatgtgtataagagacag | 263 |
| 13.10 | GCTGATCCATTAYTCA | |
| mTRBV29 | tcgtcggcagcgtcagatgtgtataagagacag | 264 |
| | CTGATTTATATCTCATACG | |
| mTRBV8 | tcgtcggcagcgtcagatgtgtataagagacag | 265 |
| | AGTAATCTAGTATTCCTA | |
| mTRBV3 | tcgtcggcagcgtcagatgtgtataagagacag | 266 |
| | GTTTCTGGTTAATTTCTAC | |
| mTRBV12 | tcgtcggcagcgtcagatgtgtataagagacag | 2 67 |
| | TTCYTYATTCAGMATTATG | |
| mTRBV | tcgtcggcagcgtcagatgtgtataagagacag | 268 |
| 22.26 | T TTCARMAT CAAGAAG | |
| mTRBV24 | tcgtcggcagcgtcagatgtgtataagagacag | 269 |
| | CGAAATGAAGAAATTATGG | |
| mTRBV | tcgtcggcagcgtcagatgtgtataagagacag | 270 |
| 23.16 | GTYCCTGAYYTACTTTC | |
| mTRBV15 | tcgtcggcagcgtcagatgtgtataagagacag | 271 |
| | GTTGCTGAGCTACTTC | |
| mTRBV14 | tcgtcggcagcgtcagatgtgtataagagacag | 272 |
| | CTTCTAGTTTACTTTCGG | |
| mTRBV21 | tcgtcggcagcgtcagatgtgtataagagacag | 273 |
| | GGTTTACTTTCAGAATGA | |
| mTRBV6 | tcgtcggcagcgtcagatgtgtataagagacag | 274 |
| | TGGCTTCATTCTATGATAC | |
| mTRBV11 | tcgtcggcagcgtcagatgtgtataagagacag | 275 |
| | TCTAACTTATTTACAGAATGG | |
| mTRBV5 | togtcggcagcgtcagatgtgtataagagacag | 27 6 |
| | GCTCATGTTTCTCTAC | |
| mTRBV4 | tcgtcggcagcgtcagatgtgtataagagacag | 277 |
| | TTCCAAGGCGCTTCTC | |
| mTRBV2 | tcgtcggcagcgtcagatgtgtataagagacag | 278 |
| | GGACAATCAGACTGCC | |
| mTRBV19 | tcgtcggcagcgtcagatgtgtataagagacag | 279 |
| | GGCTATGATGCGTCTC | |
| mTRBV17 | tcgtcggcagcgtcagatgtgtataagagacag | 280 |
| | CAGGGAAGCTGACAC | |
| mTRBV9 | tcgtcggcagcgtcagatgtgtataagagacag | 281 |
| | GTTTCTGGTGAATTTCCA | |
| mTRBV20 | tcgtcggcagcgtcagatgtgtataagagacag | 282 |
| | ATAGCACTTTCTACTGTG | |
| mTRBV31 | tcgtcggcagcgtcagatgtgtataagagacag | 283 |
| | ACTGTTGGCCAGGTAG | |
| mTRBV7 | tcgtcggcagcgtcagatgtgtataagagacag | 284 |
| | CCCCAGTTCAATACTAC | |
| mTRBV28 | tcgtcggcagcgtcagatgtgtataagagacag | 285 |
| | GGCTTATTACTCAATTAATG | |
| mTRBV1 | tcgtcggcagcgtcagatgtgtataagagacag | 286 |
| | GCTGTTCACTCTGCG | |
| mTRBV18 | tcgtcggcagcgtcagatgtgtataagagacag | 287 |
| | TGAAATTTGTGACTTCTG | |
| mTRBV30 | tcgtcggcagcgtcagatgtgtataagagacag | 288 |
| | TCATGGCAACTGCAAATG | |
| mTRBV27 | tcgtcggcagcgtcagatgtgtataagagacag | 289 |
| | GCGCCATTGTTCATATG | |

Reverse

| | | |
|---|---|---|
| A-mTRBC | gtctcgtgggctcggagatgtgtataagagacag | 290 |
| | cgtgatCAAACAAGGAGACCTTG | |
| B-mTRBC | gtctcgtgggctcggagatgtgtataagagacag | 291 |
| | acatcgCAAACAAGGAGACCTTG | |
| C-mTRBC | gtctcgtgggctcggagatgtgtataagagacag | 292 |
| | gcctaaCAAACAAGGAGACCTTG | |
| D-mTRBC | gtctcgtgggctcggagatgtgtataagagacag | 293 |
| | tggtcaCAAACAAGGAGACCTTG | |
| E-mTRBC | gtctcgtgggctcggagatgtgtataagagacag | 294 |
| | attggcCAAACAAGGAGACCTTG | |
| F-mTRBC | gtctcgtgggctcggagatgtgtataagagacag | 295 |
| | gatctgCAAACAAGGAGACCTTG | |
| G-mTRBC | gtctcgtgggctcggagatgtgtataagagacag | 296 |
| | tcaagtCAAACAAGGAGACCTTG | |
| H-mTRBC | gtctcgtgggctcggagatgtgtataagagacag | 297 |
| | ctgatcCAAACAAGGAGACCTTG | |
| I-mTRBC | gtctcgtgggctcggagatgtgtataagagacag | 298 |
| | aagctaCAAACAAGGAGACCTTG | |
| J-mTRBC | gtctcgtgggctcggagatgtgtataagagacag | 299 |
| | gtagccCAAACAAGGAGACCTTG | |
| K-mTRBC | gtctcgtgggctcggagatgtgtataagagacag | 300 |
| | tacaagCAAACAAGGAGACCTTG | |
| L-mTRBC | gtctcgtgggctcggagatgtgtataagagacag | 301 |
| | cactgtCAAACAAGGAGACCTTG | |
| M-mTRBC | gtctcgtgggctcggagatgtgtataagagacag | 302 |
| | ttgactCAAACAAGGAGACCTTG | |
| N-mTRBC | gtctcgtgggctcggagatgtgtataagagacag | 303 |
| | ggaactCAAACAAGGAGACCTTG | |
| O-mTRBC | gtctcgtgggctcggagatgtgtataagagacag | 304 |
| | tga catCAAACAAGGAGACCTTG | |
| P-mTRBC | gtctcgtgggctcggagatgtgtataagagacag | 305 |
| | ggacggCAAACAAGGAGACCTTG | |
| Q-mTRBC | gtctcgtgggctcggagatgtgtataagagacag | 306 |
| | ctctacCAAACAAGGAGACCTTG | |
| R-mTRBC | gtctcgtgggctcggagatgtgtataagagacag | 307 |
| | gcggacCAAACAAGGAGACCTTG | |

Primers targeting mTRBV genes are sense.
Primers targeting mTRBC genes are antisense.
mTRBV, mouse T cell receptor Vβ; mTRBC, mouse T cell receptor cβ.
Lower case letters, adapter sequences (ILLUMINA®).
Bold and underlined sequences, barcode sequences (ILLUMINA®).

Stocks of primers were prepared by resuspending the primers to 200 μM using 1×TE buffer (low EDTA; 10 mM Tris-HCl, 0.1 mM EDTA, pH 8.0) and stored at −20° C.

Cocktails of external forward human or mouse TRAV (TRAV EXT FOR) and internal forward human or mouse TRAV (TRAV INT FOR) primers were prepared by combining 25 μl of each human or mouse TRAV primer (40 of them, 200 μM stock), thereby yielding 1000 μl of diluted primer cocktail with each primer at 5 μM working concentration.

Cocktails of external forward human or mouse TRBV (TRBV EXT FOR) and internal forward human or mouse TRBV (TRBV INT FOR) primers was prepared by combining 25 μl of each human or mouse TRBV EXT forward primer (27 of them, 200 μM stock) with 325 μl 1×TE buffer (low EDTA; 10 mM Tris-HCl, 0.1 mM EDTA, pH 8.0), thereby yielding 1000 μl of diluted primer cocktail with each primer at 5 μM working concentration.

All reverse primers including human or mouse TRAC external reverse (TRAC EXT REV), human or mouse TRBC external reverse (TRBC EXT REV), human or mouse TRAC internal reverse (TRAC INT REV), human or mouse TRBC internal reverse (TRBC INT REV) were resuspended to prepare 5 μM working stocks.

Single Cell Sorting. Mouse or human CD8+ T cells isolated from peripheral blood mononuclear cells (PBMC), Bronchoalveolar lavage (BAL), spleen (SPL), and lymph nodes (LN) of infected, naïve or memory mice or humans. Cells were resuspended in 1 ml of Dulbecco's phosphate-buffered saline (D-PBS) without Ca⁺⁺/Mg⁺⁺ or extraneous proteins. The cells were stained with LIVE/DEAD® Fixable Aqua Dead Cell Stain (405 nm excitation) discrimination dye for 30 minutes at room temperature in the dark. At the end of the incubation, cells were washed twice with sort buffer (PBS containing 0.1% BSA, fraction V (Life Technologies)) by centrifugation at 500 g and +4° C. for 5 minutes.

Cells were subsequently resuspended in 50 μl of sort buffer containing blocking antibody (anti-mouse CD16/CD32) or APC-conjugated peptide-loaded pMHCI tetramer (e.g., HLA-A*0201-CMV pp65; Beckman Coulter) in appropriate dilution and incubated at room temperature in the dark for 1 hour. The cells were again washed twice by centrifuging at 500 g and +4° C. for 5 minutes using sort buffer. For human cells, the cell pellet was resuspended in sort buffer containing an appropriate dilution of FITC-conjugated anti-human CD3, PE-Cy7-conjugated anti-human CD8, PE-conjugated anti-human CD14. For mouse cells, the cell pellet was resuspended in sort buffer containing an appropriate dilution of mouse-CD4, anti-mouse CD11b, anti-mouse CD11c, F4/80 (all Pacific Blue-conjugated for negative gating)(Biolegend), and anti-mouse CD8-APC-eFluro780 (eBiosciences). Subsequently, the cells were incubated on ice for 20 minutes in the dark.

Cells were again washed twice by centrifuging at 500 g and +4° C. for 5 minutes using sort buffer. The resulting pellets were resuspending in 0.5 ml of sort buffer containing RNase inhibitor at 200 U/ml. The cell suspension was filtered through a 40 μM cell strainer. Lymphocytes were first gated on their scatter properties based on a FSC-A/SSC-A plot. From the probable lymphocyte population, the live cells were select based on Fixable LIVE/DEAD® aqua staining and CD3, CD8, and CD14 negative cells for human cells, or CD4, CD11b, CD11c and F4/80 negative cells for mouse cells. Cells were than gated on CDB+ tetramer+ for sorting.

Epitope-specific CD8 cells were sorted into each well of columns 1-23 of a 384-well polypropylene PCR plate (Eppendorf). Column 24 of the 384-well plate was left empty for the negative (no template) control. Following the sort, the plate was sealed with adhesive plate seal (MicroAmp, Applied Biosystem) and placed on ice. The plates were briefly spun to bring the contents down and frozen at −80° C.

Reverse transcription (RT). The reverse transcription of the TCRα and β mRNA was carried out directly on the lysed cells without any RNA extraction step. Lysis was achieved by a combination of the freeze-thaw cycle and inclusion of a detergent (Triton™ X-100, 0.1% final) in the RT mixture. Either iScript™ (Biorad) or SuperScript® Vilo™ (Thermo) were used for RT as follows. More specifically, the plate was thawed, centrifuged at 500 g for 2 minutes and keep on ice. RT master mixes were prepared as appropriate for the iScript™ (Biorad) and SuperScript® Vilo™ enzymes (Table 9).

TABLE 9

| | | |
|---|---|---|
| | Per well | One Plate |
| Component | (Total 1 μl) | (450 reactions) |

iScript ™ (384-well plate)

| | | |
|---|---|---|
| 5× RT Buffer | 0.2 μl | 90 μl |
| iScript ™ RT enzyme | 0.05 μl | 22.5 μl |
| Water, Nuclease free | 0.65 μl | 292.5 μl |
| Triton-X100 (1%) | 0.1 μl | 45 μl |

SuperScript ® Vilo ™ (384-well plate)

| | | |
|---|---|---|
| 5× RT Buffer | 0.2 μl | 90 μl |
| 10× SuperScript ® RT enzyme | 0.1 μl | 45 μl |
| Water, Nuclease free | 0.6 μl | 270 μl |
| Triton-X100 (1%) | 0.1 μl | 45 μl |

To each well was added one μl of RT master mix using a multichannel pipette. Once all columns were filled, the plate was resealed and centrifuged at 500 g for 2 minutes. Using a thermocycler, cDNA was synthesized as follows: 5 minutes at 25° C.; 45 minutes at 42° C.; 5 minutes at 85° C.; hold at 4° C. After cDNA synthesis, the plate was stored at −20° C.

Frist Round of Polymerase chain reaction (PCR). Nested PCR was carried out to amplify the TCR α and β chain from the single cells. The reaction mixture, which included the primers listed in Tables 1 and 2 (human) or Tables 5 and 6 (mouse), for the first round PCR (384 well plate) is provided in Table 10.

TABLE 10

| | | One Plate |
|---|---|---|
| Component | Per well | (450 reactions) |
| Water, Nuclease free | 6.9 μl | 3105 μl |
| PCR buffer, 10× | 1 μl | 450 μl |
| (Containing 15 mM MgCl₂) | | |
| dNTP, 10 mM | 0.2 μl | 90 μl |
| TRAC EXT REV (5-20 μM) | 0.2 μl | 90 μl |
| TRAV-EXT FOR | 0.2 μl | 90 μl |
| (Cocktail α primers) | | |
| TRBC EXT REV (5-20 μM) | 0.2 μl | 90 μl |
| TRBV EXT FOR | 0.2 μl | 90 μl |
| (Cocktail β primers) | | |
| Taq DNA Polymerase | 0.1 μl | 45 μl |
| Total | 9 μl | 4050 μl |

To each sample and control well of the cDNA plate was add 9 μl of the master mix. The plate was resealed and centrifuged to bring the contents to the bottom of the wells. PCR of human TCRαβ was carried out in a thermocycler as follows: initial denaturation at 95° C. for 5 minutes; 34 cycles of denaturation at 95° C. for 20 seconds, primer annealing at 52° C. for 20 seconds, polymerase extension at 72° for 45 seconds; a final extension at 72° C. for 7 minutes and a final hold at 4° C. PCR of mouse TCRαβ was carried out as described for human TCRαβ except that primer annealing was carried out at 55° C. instead of 52° C. The plates were stored at −20° C. until the next step.

Nested PCR. TCR alpha and beta nested PCR reactions were set up separately. Accordingly, identical wells of alpha and beta plates are from a single cell. For example, well position A1 of the alpha plate will pair with A1 of the beta plate (from a single cell). The primer set for internal PCR for next generation sequencing platform is distinct from Sanger's sequencing method. Each plate is barcoded with a unique reverse primer (6 nucleotide barcode). Thus, each plate is labeled with the barcode name (A, B, C, etc.) so that multiple plates can be pooled for the sequencing run.

Two master mixes (alpha and beta) were prepared as provided in Table 11 for the second round PCR (384-well plate).

TABLE 11

| | | One Plate |
|---|---|---|
| Component | Per well | (450 reactions) |
| Water, Nuclease free | 7.3 μl | 3285 μl |
| CoralLoad PCR buffer, 10× | 1 μl | 450 μl |
| (Containing 15 mM MgCl₂) | | |
| dNTP, 10 mM | 0.2 μl | 90 μl |
| TRAC INT REV (5-20 μM, | 0.2 μl | 90 μl |
| Table 3 or Table 7) | | |
| or | | |
| TRBC INT REV (5-20 μM, | | |
| Table 4 or Table 8) | | |
| TRAC INT FOR (Cocktail of | 0.2 μl | 90 μl |
| α primers, Table 3 or | | |
| Table 7) | | |
| or | | |
| TRBC INT FOR (Cocktail of | | |
| β primers, Table 4 or | | |
| Table 8) | | |
| Tag DNA Polymerase | 0.1 μl | 45 μl |
| Total | 9 μl | 4050 μl |

To each well of the alpha and beta plates was add 9 μl of the master mix and 1 μl of the amplicons from the first round. The plate was resealed and centrifuged to bring the contents to the bottom of the wells. PCR of human TCRαβ was carried out in a thermocycler as follows: initial denaturation at 95° C. for 5 minutes; 34 cycles of denaturation at 95° C. for 20 seconds, primer annealing at 52° C. for 20 seconds, polymerase extension at 72° for 45 seconds; a final extension at 72° C. for 7 minutes and a final hold at 4° C. PCR of mouse TCRαβ was carried out as described for human TCRαβ except that primer annealing was carried out at 55° C. instead of 52° C. Samples in the plates were immediately analyzed by agarose gel (2% in 1×TAE buffer) electrophoresis (2.5 μl) and stored at −80° C.

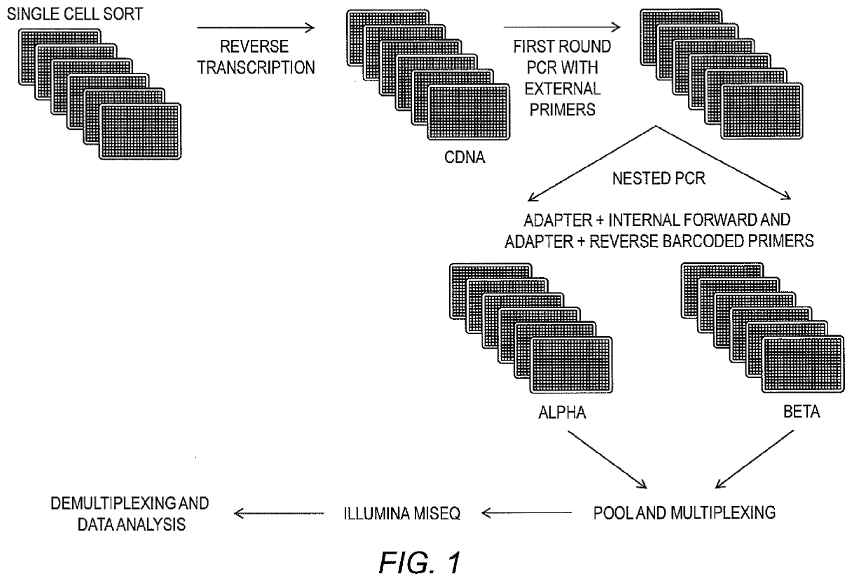

Sequencing. Following PCR, the plates were stored at −80° C. until all the intended plates were amplified. All the 384-well plates (with different barcoded reverse primers) were pooled into a single 384-well plate using an Integra Viaflo pipetting system. Amplicons were sequenced on an ILLUMINA® MiSeq platform, which uses reversible-terminator sequencing by synthesis technology. A schematic of the single cell paired TCR deep sequencing method of the invention is presented in FIG. 1.

### Example 2: Materials and Methods

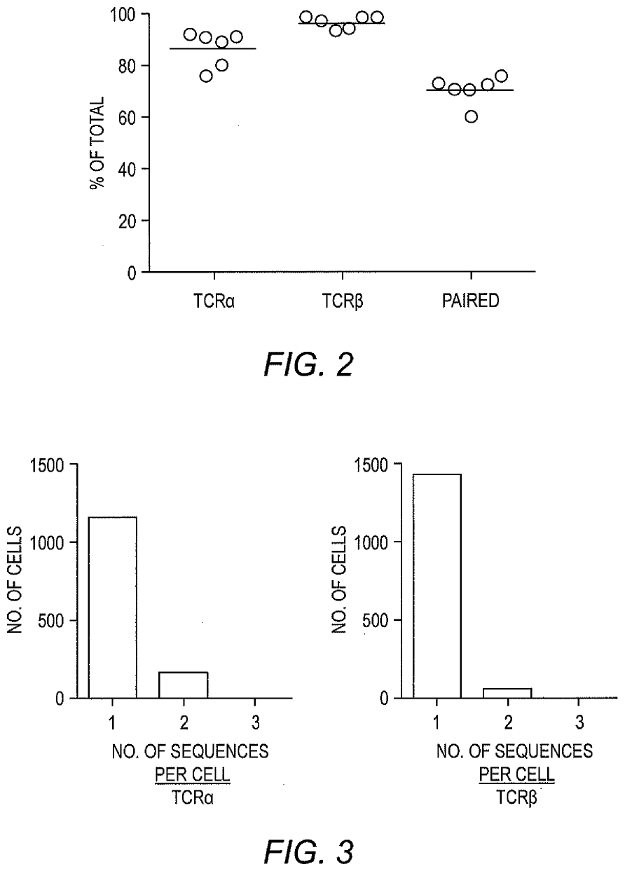

Using the method of the invention, a single cell paired TCR deep sequencing experiment was conducted. The results of this analysis indicated an increase in paired data (FIG. 2). In addition, it was observed that most of the cells were monoallelic and approximately 10% of the cells were dual in-frame TCR α cells (FIG. 3). Notably, the method allowed for 384-well multiplexing, plate multiplexing, and single cell level paired data (˜1500 to 3000), which is efficient and economically viable as reagent use was decreased.

1: A kit for analyzing a single T cell comprising:
(a) a first set of primers, wherein the first set of primers comprises:
(i) a first set of forward primers comprising the nucleotide sequences of SEQ ID NOs:1-40 and SEQ ID NOs:42-70 having a length ranging from 15 to 40 nucleotides,
(ii) a first set of reverse primers comprising the nucleotide sequences of SEQ ID NOs:41 and 71 having a length ranging from 15 to 40 nucleotides; and
(b) a second set of primers, wherein the second set of primers comprises:
(i) a second set of forward primers comprising the nucleotide sequences of SEQ ID NOs:72-111 and SEQ ID NO:130-158 having a length ranging from 40 to 70 nucleotides, and
(ii) a second set of reverse primers comprising the nucleotide sequences of SEQ ID NOs:112-129 and SEQ ID NOs:159-176 having a length ranging from 50 to 90 nucleotides;
or
(c) a first set of primers, wherein the first set of primers comprises:
(i) a first set of forward primers comprising the nucleotide sequences of SEQ ID NOs:177-199 and SEQ ID NOs:201-217 having a length ranging from 15 to 40 nucleotides,
(ii) a first set of reverse primers comprising the nucleotide sequences of SEQ ID NOs:200 and 218 having a length ranging from 15 to 40 nucleotides; and
(d) a second set of primers, wherein the second set of primers comprises:
(i) a second set of forward primers comprising the nucleotide sequences of SEQ ID NOs:219-244 and SEQ ID NO:263-289 having a length ranging from 40 to 70 nucleotides, and
(ii) a second set of reverse primers comprising the nucleotide sequences of SEQ ID NOs:245-262 and SEQ ID NOs:290-307 having a length ranging from 50 to 90 nucleotides.

2: A method for analyzing nucleic acid molecules encoding T cell receptor (TCR) α and β from single T cells, comprising:
(a) sorting single T cells from a sample comprising a plurality of T cells into separate locations;
(b) amplifying nucleic acid molecules encoding TCRα and TCRβ from one or more single T cells using the first set of primers from the kit of claim 1 to produce a first set of amplicon products in one or more locations of the separate locations;
(c) performing nested polymerase chain reaction (PCR) on the amplified nucleic acid molecules encoding TCRα and TCRβ in the first set of amplicon products with the second set of primers from the kit of claim 1 to produce a second set of amplicon products; and
(d) sequencing the amplicon products.

3: The method of claim 2, wherein the sequencing step (d) comprises:
(i) carrying out a third round of PCR to produce a third set of amplicon products, and
(ii) subjecting the third amplicon products to next generation sequencing.

4: The method of claim 2, wherein the nucleic acid molecules encoding TCRα and TCRβ are mRNAs.

5: The method of claim 2, wherein the sample is collected from a subject.

6. (canceled)


SEQUENCE LISTING (309 sequences)

SEQ ID NO: 1 - DNA, 20 bp, Artificial Sequence
  Synthetic oligonucleotide.
aactgcacgtaccagacatc

SEQ ID NO: 2 - DNA, 18 bp, Artificial Sequence
  Synthetic oligonucleotide.
gatgtgcaccaagactcc

SEQ ID NO: 3 - DNA, 19 bp, Artificial Sequence
  Synthetic oligonucleotide.
aagatcaggtcaacgttgc

SEQ ID NO: 4 - DNA, 21 bp, Artificial Sequence
  Synthetic oligonucleotide.
ctccatggactcatatgaagg

SEQ ID NO: 5 - DNA, 19 bp, Artificial Sequence
  Synthetic oligonucleotide.
cttttcctgagtgtccgag

SEQ ID NO: 6 - DNA, 21 bp, Artificial Sequence
  Synthetic oligonucleotide.
caccctgacctgcaactatac

SEQ ID NO: 7 - DNA, 18 bp, Artificial Sequence
  Synthetic oligonucleotide.
gcaaaatacagggatggg

SEQ ID NO: 8 - DNA, 18 bp, Artificial Sequence
  Synthetic oligonucleotide.
ctcactggagttgggatg

SEQ ID NO: 9 - DNA, 19 bp, Artificial Sequence
  Synthetic oligonucleotide.
cactgtctctgaaggagcc

SEQ ID NO: 10 - DNA, 17 bp, Artificial Sequence
  Synthetic oligonucleotide.
gccaccctggttaaagg

SEQ ID NO: 11 - DNA, 20 bp, Artificial Sequence
  Synthetic oligonucleotide.
gagctgaggtgcaactactc

SEQ ID NO: 12 - DNA, 18 bp, Artificial Sequence
  Synthetic oligonucleotide.
ctaacagaggccacccag

SEQ ID NO: 13 - DNA, 20 bp, Artificial Sequence
  Synthetic oligonucleotide.
tggtatgtccaatatcctgg

SEQ ID NO: 14 - DNA, 19 bp, Artificial Sequence
  Synthetic oligonucleotide.
caagtggagcagagtcctc

SEQ ID NO: 15 - DNA, 18 bp, Artificial Sequence
  Synthetic oligonucleotide.
cartgttccagagggagc

SEQ ID NO: 16 - DNA, 19 bp, Artificial Sequence
  Synthetic oligonucleotide.
catccttcaaccctgagtg

SEQ ID NO: 17 - DNA, 19 bp, Artificial Sequence
  Synthetic oligonucleotide.
cagcgcctcagactacttc

SEQ ID NO: 18 - DNA, 22 bp, Artificial Sequence
  Synthetic oligonucleotide.
aagataactcaaacccaaccag

SEQ ID NO: 19 - DNA, 19 bp, Artificial Sequence
  Synthetic oligonucleotide.
agtggagctgaagtgcaac

SEQ ID NO: 20 - DNA, 19 bp, Artificial Sequence
  Synthetic oligonucleotide.
ggagaagaggatcctcagg

SEQ ID NO: 21 - DNA, 22 bp, Artificial Sequence
  Synthetic oligonucleotide.
tccagtatctaaacaaagagcc

SEQ ID NO: 22 - DNA, 19 bp, Artificial Sequence
  Synthetic oligonucleotide.
aggtaactcaagcgcagac

SEQ ID NO: 23 - DNA, 20 bp, Artificial Sequence
  Synthetic oligonucleotide.
cacagtcagcggtttaagag

SEQ ID NO: 24 - DNA, 18 bp, Artificial Sequence
  Synthetic oligonucleotide.
ttcctgcagctctgagtg

SEQ ID NO: 25 - DNA, 20 bp, Artificial Sequence
  Synthetic oligonucleotide.
gtcctccagacctgattctc

SEQ ID NO: 26 - DNA, 19 bp, Artificial Sequence
  Synthetic oligonucleotide.
tgcttatgagaacactgcg

SEQ ID NO: 27 - DNA, 20 bp, Artificial Sequence
  Synthetic oligonucleotide.
ctcagtcactgcatgttcag

SEQ ID NO: 28 - DNA, 19 bp, Artificial Sequence
  Synthetic oligonucleotide.
ggacttcaccacgtactgc

SEQ ID NO: 29 - DNA, 19 bp, Artificial Sequence
  Synthetic oligonucleotide.
gcaaacctgccttgtaatc

SEQ ID NO: 30 - DNA, 19 bp, Artificial Sequence
  Synthetic oligonucleotide.
agccaaattcaatggagag

SEQ ID NO: 31 - DNA, 22 bp, Artificial Sequence
  Synthetic oligonucleotide.
tcagtttctaagcatccaagag

SEQ ID NO: 32 - DNA, 21 bp, Artificial Sequence
  Synthetic oligonucleotide.
gcaagttaagcaaaattcacc

SEQ ID NO: 33 - DNA, 19 bp, Artificial Sequence
  Synthetic oligonucleotide.
caacaaccagtgcagagtc

SEQ ID NO: 34 - DNA, 20 bp, Artificial Sequence
  Synthetic oligonucleotide.
agaactggagcagagtcctc

SEQ ID NO: 35 - DNA, 20 bp, Artificial Sequence
  Synthetic oligonucleotide.
ggtcaacagctgaatcagag

SEQ ID NO: 36 - DNA, 21 bp, Artificial Sequence
  Synthetic oligonucleotide.
gaagacaaggtggtacaaagc

SEQ ID NO: 37 - DNA, 20 bp, Artificial Sequence
  Synthetic oligonucleotide.
gcacatatgacaccagtgag

SEQ ID NO: 38 - DNA, 18 bp, Artificial Sequence
  Synthetic oligonucleotide.
ctgttcctgagcatgcag

SEQ ID NO: 39 - DNA, 21 bp, Artificial Sequence
  Synthetic oligonucleotide.
gcatctgtgactatgaactgc

SEQ ID NO: 40 - DNA, 20 bp, Artificial Sequence
  Synthetic oligonucleotide.
aatgaagtggagcagagtcc

SEQ ID NO: 41 - DNA, 19 bp, Artificial Sequence
  Synthetic oligonucleotide.
gaccagcttgacatcacag

SEQ ID NO: 42 - DNA, 21 bp, Artificial Sequence
  Synthetic oligonucleotide.
tcgatgatcaattctcagttg

SEQ ID NO: 43 - DNA, 20 bp, Artificial Sequence
  Synthetic oligonucleotide.
caaaatacctggtcacacag

SEQ ID NO: 44 - DNA, 18 bp, Artificial Sequence
  Synthetic oligonucleotide.
tcgcttctcacctgaatg

SEQ ID NO: 45 - DNA, 20 bp, Artificial Sequence
  Synthetic oligonucleotide.
gattctcaggkckccagttc

SEQ ID NO: 46 - DNA, 19 bp, Artificial Sequence
  Synthetic oligonucleotide.
gtaccaacaggycctgggt

SEQ ID NO: 47 - DNA, 19 bp, Artificial Sequence
  Synthetic oligonucleotide.
actcagaccccaaaattcc

SEQ ID NO: 48 - DNA, 19 bp, Artificial Sequence
  Synthetic oligonucleotide.
actggcaaaggagaagtcc

SEQ ID NO: 49 - DNA, 20 bp, Artificial Sequence
  Synthetic oligonucleotide.
trtgatccaatttcaggtca

SEQ ID NO: 50 - DNA, 19 bp, Artificial Sequence
  Synthetic oligonucleotide.
cgswtctytgcagaraggc

SEQ ID NO: 51 - DNA, 19 bp, Artificial Sequence
  Synthetic oligonucleotide.
gatcacagcaactggacag

SEQ ID NO: 52 - DNA, 18 bp, Artificial Sequence
  Synthetic oligonucleotide.
cagagcccaagacacaag

SEQ ID NO: 53 - DNA, 20 bp, Artificial Sequence
  Synthetic oligonucleotide.
accttgatgtgtcaccagac

SEQ ID NO: 54 - DNA, 18 bp, Artificial Sequence
  Synthetic oligonucleotide.
cagagcccaagacacaag

SEQ ID NO: 55 - DNA, 19 bp, Artificial Sequence
  Synthetic oligonucleotide.
cgattttctgcagagacgc

SEQ ID NO: 56 - DNA, 20 bp, Artificial Sequence
  Synthetic oligonucleotide.
argtgacagaratgggacaa

SEQ ID NO: 57 - DNA, 19 bp, Artificial Sequence
  Synthetic oligonucleotide.
agcgataaaggaagcatcc

SEQ ID NO: 58 - DNA, 21 bp, Artificial Sequence
  Synthetic oligonucleotide.
ccaacaatcgattcttagctg

SEQ ID NO: 59 - DNA, 20 bp, Artificial Sequence
  Synthetic oligonucleotide.
agtgaccctgagttgttctc

SEQ ID NO: 60 - DNA, 22 bp, Artificial Sequence
  Synthetic oligonucleotide.
gtctttgatgaaacaggtatgc

SEQ ID NO: 61 - DNA, 18 bp, Artificial Sequence
  Synthetic oligonucleotide.
cagacccccagacacaag

SEQ ID NO: 62 - DNA, 21 bp, Artificial Sequence
  Synthetic oligonucleotide.
catagatgagtcaggaatgcc

SEQ ID NO: 63 - DNA, 22 bp, Artificial Sequence
  Synthetic oligonucleotide.
agttgtgaacagaatttgaacc

SEQ ID NO: 64 - DNA, 20 bp, Artificial Sequence
  Synthetic oligonucleotide.
aagtttctcatcaaccatgc

SEQ ID NO: 65 - DNA, 19 bp, Artificial Sequence
  Synthetic oligonucleotide.
gcgattctcatctcaatgc

SEQ ID NO: 66 - DNA, 22 bp, Artificial Sequence
  Synthetic oligonucleotide.
cctacggttgatctattactcc

SEQ ID NO: 67 - DNA, 22 bp, Artificial Sequence
  Synthetic oligonucleotide.
actacacctcatccactattcc

SEQ ID NO: 68 - DNA, 19 bp, Artificial Sequence
  Synthetic oligonucleotide.
tggtatcgacaagacccag

SEQ ID NO: 69 - DNA, 19 bp, Artificial Sequence
  Synthetic oligonucleotide.
ttctggtaccgtcagcaac

SEQ ID NO: 70 - DNA, 20 bp, Artificial Sequence
  Synthetic oligonucleotide.
tccagctgctcttctactcc

SEQ ID NO: 71 - DNA, 20 bp, Artificial Sequence
  Synthetic oligonucleotide.
tagaactggacttgacagcg

SEQ ID NO: 72 - DNA, 54 bp, Artificial Sequence
  Synthetic oligonucleotide.
tcgtcggcagcgtcagatgtgtataagagacaggcacccacatttctktcttac

SEQ ID NO: 73 - DNA, 50 bp, Artificial Sequence
  Synthetic oligonucleotide.
tcgtcggcagcgtcagatgtgtataagagacagccttctcagcagggacg

SEQ ID NO: 74 - DNA, 50 bp, Artificial Sequence
  Synthetic oligonucleotide.
tcgtcggcagcgtcagatgtgtataagagacagaccgaggcctccagttc

SEQ ID NO: 75 - DNA, 52 bp, Artificial Sequence
  Synthetic oligonucleotide.
tcgtcggcagcgtcagatgtgtataagagacagagtggcctccctgtttatc

SEQ ID NO: 76 - DNA, 55 bp, Artificial Sequence
  Synthetic oligonucleotide.
tcgtcggcagcgtcagatgtgtataagagacagccaaagactcactgttctattg

SEQ ID NO: 77 - DNA, 52 bp, Artificial Sequence
  Synthetic oligonucleotide.
tcgtcggcagcgtcagatgtgtataagagacagacaagatccaggaagaggc

SEQ ID NO: 78 - DNA, 55 bp, Artificial Sequence
  Synthetic oligonucleotide.
tcgtcggcagcgtcagatgtgtataagagacagtatgagaagcagaaaggaagac

SEQ ID NO: 79 - DNA, 53 bp, Artificial Sequence
  Synthetic oligonucleotide.
tcgtcggcagcgtcagatgtgtataagagacaggtcaacaccttcagcttctc

SEQ ID NO: 80 - DNA, 53 bp, Artificial Sequence
  Synthetic oligonucleotide.
tcgtcggcagcgtcagatgtgtataagagacagagagtgaaacctccttccac

SEQ ID NO: 81 - DNA, 54 bp, Artificial Sequence
  Synthetic oligonucleotide.
tcgtcggcagcgtcagatgtgtataagagacagtttgaggctgaatttaagagg

SEQ ID NO: 82 - DNA, 52 bp, Artificial Sequence
  Synthetic oligonucleotide.
tcgtcggcagcgtcagatgtgtataagagacagaaccaaggactccagcttc

SEQ ID NO: 83 - DNA, 52 bp, Artificial Sequence
  Synthetic oligonucleotide.
tcgtcggcagcgtcagatgtgtataagagacagatcagaggttttgaggctg

SEQ ID NO: 84 - DNA, 54 bp, Artificial Sequence
  Synthetic oligonucleotide.
tcgtcggcagcgtcagatgtgtataagagacaggaaaccacttctttccacttg

SEQ ID NO: 85 - DNA, 54 bp, Artificial Sequence
  Synthetic oligonucleotide.
tcgtcggcagcgtcagatgtgtataagagacaggaagatatacagcaactctgg

SEQ ID NO: 86 - DNA, 54 bp, Artificial Sequence
  Synthetic oligonucleotide.
tcgtcggcagcgtcagatgtgtataagagacagaagatggaaggtttacagcac

SEQ ID NO: 87 - DNA, 55 bp, Artificial Sequence
  Synthetic oligonucleotide.
tcgtcggcagcgtcagatgtgtataagagacagccaacgaattgctgttacattg

SEQ ID NO: 88 - DNA, 54 bp, Artificial Sequence
  Synthetic oligonucleotide.
tcgtcggcagcgtcagatgtgtataagagacagcagtgaaacatctctctctgc

SEQ ID NO: 89 - DNA, 52 bp, Artificial Sequence
  Synthetic oligonucleotide.
tcgtcggcagcgtcagatgtgtataagagacagcaacagaaggtcgctactc

SEQ ID NO: 90 - DNA, 53 bp, Artificial Sequence
  Synthetic oligonucleotide.
tcgtcggcagcgtcagatgtgtataagagacaggtccagtactccagacaacg

SEQ ID NO: 91 - DNA, 54 bp, Artificial Sequence
  Synthetic oligonucleotide.
tcgtcggcagcgtcagatgtgtataagagacagcagtggaagattaagagtcac

SEQ ID NO: 92 - DNA, 52 bp, Artificial Sequence
  Synthetic oligonucleotide.
tcgtcggcagcgtcagatgtgtataagagacagtgacagttccttccacctg

SEQ ID NO: 93 - DNA, 53 bp, Artificial Sequence
  Synthetic oligonucleotide.
tcgtcggcagcgtcagatgtgtataagagacaggtggtcggtattcttggaac

SEQ ID NO: 94 - DNA, 53 bp, Artificial Sequence
  Synthetic oligonucleotide.
tcgtcggcagcgtcagatgtgtataagagacagctcttcaccctgtattcagc

SEQ ID NO: 95 - DNA, 53 bp, Artificial Sequence
  Synthetic oligonucleotide.
tcgtcggcagcgtcagatgtgtataagagacaggtggaagacttaatgcctcg

SEQ ID NO: 96 - DNA, 50 bp, Artificial Sequence
  Synthetic oligonucleotide.
tcgtcggcagcgtcagatgtgtataagagacaggccacgactgtcgctac

SEQ ID NO: 97 - DNA, 54 bp, Artificial Sequence
  Synthetic oligonucleotide.
tcgtcggcagcgtcagatgtgtataagagacagtgcattattgatagccatacg

SEQ ID NO: 98 - DNA, 52 bp, Artificial Sequence
  Synthetic oligonucleotide.
tcgtcggcagcgtcagatgtgtataagagacagggacgaataagtgccactc

SEQ ID NO: 99 - DNA, 55 bp, Artificial Sequence
  Synthetic oligonucleotide.
tcgtcggcagcgtcagatgtgtataagagacagtgacatttcagtttggagaagc

SEQ ID NO: 100 - DNA, 51 bp, Artificial Sequence
  Synthetic oligonucleotide.
tcgtcggcagcgtcagatgtgtataagagacagcgacagattcactcccag

SEQ ID NO: 101 - DNA, 50 bp, Artificial Sequence
  Synthetic oligonucleotide.
tcgtcggcagcgtcagatgtgtataagagacaggcctctctggcaatcgc

SEQ ID NO: 102 - DNA, 54 bp, Artificial Sequence
  Synthetic oligonucleotide.
tcgtcggcagcgtcagatgtgtataagagacaggaagctgaagagactaacctt

SEQ ID NO: 103 - DNA, 53 bp, Artificial Sequence
  Synthetic oligonucleotide.
tcgtcggcagcgtcagatgtgtataagagacagctgctgaaggtcctacattc

SEQ ID NO: 104 - DNA, 51 bp, Artificial Sequence
  Synthetic oligonucleotide.
tcgtcggcagcgtcagatgtgtataagagacagagaagcatggtgaagcac

SEQ ID NO: 105 - DNA, 55 bp, Artificial Sequence
  Synthetic oligonucleotide.
tcgtcggcagcgtcagatgtgtataagagacagtcatgaaaagataactgccaag

SEQ ID NO: 106 - DNA, 52 bp, Artificial Sequence
  Synthetic oligonucleotide.
tcgtcggcagcgtcagatgtgtataagagacagtggaagactgactgctcag

SEQ ID NO: 107 - DNA, 52 bp, Artificial Sequence
  Synthetic oligonucleotide.
tcgtcggcagcgtcagatgtgtataagagacaggtcaggaagactaagtagc

SEQ ID NO: 108 - DNA, 52 bp, Artificial Sequence
  Synthetic oligonucleotide.
tcgtcggcagcgtcagatgtgtataagagacagcagcaggcagatgattctc

SEQ ID NO: 109 - DNA, 53 bp, Artificial Sequence
  Synthetic oligonucleotide.
tcgtcggcagcgtcagatgtgtataagagacaggtgttgctatcaaatggagc

SEQ ID NO: 110 - DNA, 54 bp, Artificial Sequence
  Synthetic oligonucleotide.
tcgtcggcagcgtcagatgtgtataagagacagggaggcggaaatattaaagac

SEQ ID NO: 111 - DNA, 53 bp, Artificial Sequence
  Synthetic oligonucleotide.
tcgtcggcagcgtcagatgtgtataagagacagttgtttatgctgagctcagg

SEQ ID NO: 112 - DNA, 85 bp, Artificial Sequence
  Synthetic oligonucleotide.
gtctcgtgggctcggagatgtgtataagagacagcgtgattgttraccactractggtracatttractagtracagttracctc

SEQ ID NO: 113 - DNA, 85 bp, Artificial Sequence
  Synthetic oligonucleotide.
gtctcgtgggctcggagatgtgtataagagacagacatcgtgttraccactractggtracatttractagtracagttracctc

SEQ ID NO: 114 - DNA, 85 bp, Artificial Sequence
  Synthetic oligonucleotide.
gtctcgtgggctcggagatgtgtataagagacaggcctaatgttraccactractggtracatttractagtracagttracctc

SEQ ID NO: 115 - DNA, 85 bp, Artificial Sequence
  Synthetic oligonucleotide.
gtctcgtgggctcggagatgtgtataagagacagtggtcatgttraccactractggtracatttractagtracagttracctc

SEQ ID NO: 116 - DNA, 85 bp, Artificial Sequence
  Synthetic oligonucleotide.
gtctcgtgggctcggagatgtgtataagagacagattggctgttraccactractggtracatttractagtracagttracctc

SEQ ID NO: 117 - DNA, 85 bp, Artificial Sequence
  Synthetic oligonucleotide.
gtctcgtgggctcggagatgtgtataagagacaggatctgtgttraccactractggtracatttractagtracagttracctc

SEQ ID NO: 118 - DNA, 85 bp, Artificial Sequence
  Synthetic oligonucleotide.
gtctcgtgggctcggagatgtgtataagagacagtcaagttgttraccactractggtracatttractagtracagttracctc

SEQ ID NO: 119 - DNA, 85 bp, Artificial Sequence
  Synthetic oligonucleotide.
gtctcgtgggctcggagatgtgtataagagacagctgatctgttraccactractggtracatttractagtracagttracctc

SEQ ID NO: 120 - DNA, 85 bp, Artificial Sequence
  Synthetic oligonucleotide.
gtctcgtgggctcggagatgtgtataagagacagaagctatgttraccactractggtracatttractagtracagttracctc

SEQ ID NO: 121 - DNA, 85 bp, Artificial Sequence
  Synthetic oligonucleotide.
gtctcgtgggctcggagatgtgtataagagacaggtagcctgttraccactractggtracatttractagtracagttracctc

SEQ ID NO: 122 - DNA, 85 bp, Artificial Sequence
  Synthetic oligonucleotide.
gtctcgtgggctcggagatgtgtataagagacagtacaagtgttraccactractggtracatttractagtracagttracctc

SEQ ID NO: 123 - DNA, 85 bp, Artificial Sequence
  Synthetic oligonucleotide.
gtctcgtgggctcggagatgtgtataagagacagcactgttgttraccactractggtracatttractagtracagttracctc

SEQ ID NO: 124 - DNA, 85 bp, Artificial Sequence
  Synthetic oligonucleotide.
gtctcgtgggctcggagatgtgtataagagacagttgacttgttraccactractggtracatttractagtracagttracctc

SEQ ID NO: 125 - DNA, 85 bp, Artificial Sequence
  Synthetic oligonucleotide.
gtctcgtgggctcggagatgtgtataagagacagggaacttgttraccactractggtracatttractagtracagttracctc

SEQ ID NO: 126 - DNA, 85 bp, Artificial Sequence
  Synthetic oligonucleotide.
gtctcgtgggctcggagatgtgtataagagacagtgacattgttraccactractggtracatttractagtracagttracctc

SEQ ID NO: 127 - DNA, 85 bp, Artificial Sequence
  Synthetic oligonucleotide.
gtctcgtgggctcggagatgtgtataagagacagggacggtgttraccactractggtracatttractagtracagttracctc

SEQ ID NO: 128 - DNA, 85 bp, Artificial Sequence
  Synthetic oligonucleotide.
gtctcgtgggctcggagatgtgtataagagacagctctactgttraccactractggtracatttractagtracagttracctc

SEQ ID NO: 129 - DNA, 85 bp, Artificial Sequence
  Synthetic oligonucleotide.
gtctcgtgggctcggagatgtgtataagagacaggcggactgttraccactractggtracatttractagtracagttracctc

SEQ ID NO: 130 - DNA, 53 bp, Artificial Sequence
  Synthetic oligonucleotide.
tcgtcggcagcgtcagatgtgtataagagacagttcactctgaagatccggtc

SEQ ID NO: 131 - DNA, 54 bp, Artificial Sequence
  Synthetic oligonucleotide.
tcgtcggcagcgtcagatgtgtataagagacagaatcttcacatcaattccctg

SEQ ID NO: 132 - DNA, 51 bp, Artificial Sequence
  Synthetic oligonucleotide.
tcgtcggcagcgtcagatgtgtataagagacagcctgcagccagaagactc

SEQ ID NO: 133 - DNA, 51 bp, Artificial Sequence
  Synthetic oligonucleotide.
tcgtcggcagcgtcagatgtgtataagagacagcttggagctggrsgactc

SEQ ID NO: 134 - DNA, 52 bp, Artificial Sequence
  Synthetic oligonucleotide.
tcgtcggcagcgtcagatgtgtataagagacagtctgagctgaatgtgaacg

SEQ ID NO: 135 - DNA, 52 bp, Artificial Sequence
  Synthetic oligonucleotide.
tcgtcggcagcgtcagatgtgtataagagacagatggctacaaygtmtcyag

SEQ ID NO: 136 - DNA, 54 bp, Artificial Sequence
  Synthetic oligonucleotide.
tcgtcggcagcgtcagatgtgtataagagacagtggttatagtgtctccagagc

SEQ ID NO: 137 - DNA, 53 bp, Artificial Sequence
  Synthetic oligonucleotide.
tcgtcggcagcgtcagatgtgtataagagacagtcyactctgamgwtccagcg

SEQ ID NO: 138 - DNA, 50 bp, Artificial Sequence
  Synthetic oligonucleotide.
tcgtcggcagcgtcagatgtgtataagagacagtgrmgatycagcgcaca

SEQ ID NO: 139 - DNA, 55 bp, Artificial Sequence
  Synthetic oligonucleotide.
tcgtcggcagcgtcagatgtgtataagagacaggaaacattcttgaacgattctc

SEQ ID NO: 140 - DNA, 52 bp, Artificial Sequence
  Synthetic oligonucleotide.
tcgtcggcagcgtcagatgtgtataagagacagtggtatcgacaagacctgg

SEQ ID NO: 141 - DNA, 52 bp, Artificial Sequence; identical to SEQ ID NO: 140
  Synthetic oligonucleotide.

SEQ ID NO: 142 - DNA, 53 bp, Artificial Sequence
  Synthetic oligonucleotide.
tcgtcggcagcgtcagatgtgtataagagacagggaacaccagtgactctgag

SEQ ID NO: 143 - DNA, 53 bp, Artificial Sequence
  Synthetic oligonucleotide.
tcgtcggcagcgtcagatgtgtataagagacaggactccactctcaagatcca

SEQ ID NO: 144 - DNA, 52 bp, Artificial Sequence
  Synthetic oligonucleotide.
tcgtcggcagcgtcagatgtgtataagagacagcyactctgargatccagcc

SEQ ID NO: 145 - DNA, 54 bp, Artificial Sequence
  Synthetic oligonucleotide.
tcgtcggcagcgtcagatgtgtataagagacagcattctgaactgaacatgagc

SEQ ID NO: 146 - DNA, 54 bp, Artificial Sequence
  Synthetic oligonucleotide.
tcgtcggcagcgtcagatgtgtataagagacagattctactctgaaggtgcagc

SEQ ID NO: 147 - DNA, 53 bp, Artificial Sequence
  Synthetic oligonucleotide.
tcgtcggcagcgtcagatgtgtataagagacagataacttccaatccaggagg

SEQ ID NO: 148 - DNA, 53 bp, Artificial Sequence
  Synthetic oligonucleotide.
tcgtcggcagcgtcagatgtgtataagagacagctgtagccttgagatccagg

SEQ ID NO: 149 - DNA, 52 bp, Artificial Sequence
  Synthetic oligonucleotide.
tcgtcggcagcgtcagatgtgtataagagacaggatgcccaaggaacgattc

SEQ ID NO: 150 - DNA, 53 bp, Artificial Sequence
  Synthetic oligonucleotide.
tcgtcggcagcgtcagatgtgtataagagacagcgattttctgctgaatttcc

SEQ ID NO: 151 - DNA, 53 bp, Artificial Sequence
  Synthetic oligonucleotide.
tcgtcggcagcgtcagatgtgtataagagacagttcctctcactgtgacatcg

SEQ ID NO: 152 - DNA, 53 bp, Artificial Sequence
  Synthetic oligonucleotide.
tcgtcggcagcgtcagatgtgtataagagacagactctgacagtgaccagtgc

SEQ ID NO: 153 - DNA, 52 bp, Artificial Sequence
  Synthetic oligonucleotide.
tcgtcggcagcgtcagatgtgtataagagacaggcaatcctgtcctcagaac

SEQ ID NO: 154 - DNA, 54 bp, Artificial Sequence
  Synthetic oligonucleotide.
tcgtcggcagcgtcagatgtgtataagagacaggatggatacagtgtctctcga

SEQ ID NO: 155 - DNA, 53 bp, Artificial Sequence
  Synthetic oligonucleotide.
tcgtcggcagcgtcagatgtgtataagagacagcagagaagggagatctttcc

SEQ ID NO: 156 - DNA, 53 bp, Artificial Sequence
  Synthetic oligonucleotide.
tcgtcggcagcgtcagatgtgtataagagacagttcyccctgatyctggagtc

SEQ ID NO: 157 - DNA, 54 bp, Artificial Sequence
  Synthetic oligonucleotide.
tcgtcggcagcgtcagatgtgtataagagacagtctgactgtgagcaacatgag

SEQ ID NO: 158 - DNA, 54 bp, Artificial Sequence
  Synthetic oligonucleotide.
tcgtcggcagcgtcagatgtgtataagagacagagaatctctcagcctccagac

SEQ ID NO: 159 - DNA, 60 bp, Artificial Sequence
  Synthetic oligonucleotide.
gtctcgtgggctcggagatgtgtataagagacagcgtgatttctgatggctcaaacacag

SEQ ID NO: 160 - DNA, 60 bp, Artificial Sequence
  Synthetic oligonucleotide.
gtctcgtgggctcggagatgtgtataagagacagacatcgttctgatggctcaaacacag

SEQ ID NO: 161 - DNA, 60 bp, Artificial Sequence
  Synthetic oligonucleotide.
gtctcgtgggctcggagatgtgtataagagacaggcctaattctgatggctcaaacacag

SEQ ID NO: 162 - DNA, 60 bp, Artificial Sequence
  Synthetic oligonucleotide.
gtctcgtgggctcggagatgtgtataagagacagtggtcattctgatggctcaaacacag

SEQ ID NO: 163 - DNA, 60 bp, Artificial Sequence
  Synthetic oligonucleotide.
gtctcgtgggctcggagatgtgtataagagacagattggcttctgatggctcaaacacag

SEQ ID NO: 164 - DNA, 60 bp, Artificial Sequence
  Synthetic oligonucleotide.
gtctcgtgggctcggagatgtgtataagagacaggatctgttctgatggctcaaacacag

SEQ ID NO: 165 - DNA, 60 bp, Artificial Sequence
  Synthetic oligonucleotide.
gtctcgtgggctcggagatgtgtataagagacagtcaagtttctgatggctcaaacacag

SEQ ID NO: 166 - DNA, 60 bp, Artificial Sequence
  Synthetic oligonucleotide.
gtctcgtgggctcggagatgtgtataagagacagctgatcttctgatggctcaaacacag

SEQ ID NO: 167 - DNA, 60 bp, Artificial Sequence
  Synthetic oligonucleotide.
gtctcgtgggctcggagatgtgtataagagacagaagctattctgatggctcaaacacag

SEQ ID NO: 168 - DNA, 60 bp, Artificial Sequence
  Synthetic oligonucleotide.
gtctcgtgggctcggagatgtgtataagagacaggtagccttctgatggctcaaacacag

SEQ ID NO: 169 - DNA, 60 bp, Artificial Sequence
  Synthetic oligonucleotide.
gtctcgtgggctcggagatgtgtataagagacagtacaagttctgatggctcaaacacag

SEQ ID NO: 170 - DNA, 60 bp, Artificial Sequence
  Synthetic oligonucleotide.
gtctcgtgggctcggagatgtgtataagagacagcactgtttctgatggctcaaacacag

SEQ ID NO: 171 - DNA, 60 bp, Artificial Sequence
  Synthetic oligonucleotide.
gtctcgtgggctcggagatgtgtataagagacagttgactttctgatggctcaaacacag

SEQ ID NO: 172 - DNA, 60 bp, Artificial Sequence
  Synthetic oligonucleotide.
gtctcgtgggctcggagatgtgtataagagacagggaactttctgatggctcaaacacag

SEQ ID NO: 173 - DNA, 60 bp, Artificial Sequence
  Synthetic oligonucleotide.
gtctcgtgggctcggagatgtgtataagagacagtgacatttctgatggctcaaacacag

SEQ ID NO: 174 - DNA, 60 bp, Artificial Sequence
  Synthetic oligonucleotide.
gtctcgtgggctcggagatgtgtataagagacagggacggttctgatggctcaaacacag

SEQ ID NO: 175 - DNA, 60 bp, Artificial Sequence
  Synthetic oligonucleotide.
gtctcgtgggctcggagatgtgtataagagacagctctacttctgatggctcaaacacag

SEQ ID NO: 176 - DNA, 60 bp, Artificial Sequence
  Synthetic oligonucleotide.
gtctcgtgggctcggagatgtgtataagagacaggcggacttctgatggctcaaacacag

SEQ ID NO: 177 - DNA, 19 bp, Artificial Sequence
  Synthetic oligonucleotide.
ggttatcctggtaccagca

SEQ ID NO: 178 - DNA, 20 bp, Artificial Sequence
  Synthetic oligonucleotide.
catctactggtaccgacagg

SEQ ID NO: 179 - DNA, 15 bp, Artificial Sequence
  Synthetic oligonucleotide.
ggcgagcaggtggag

SEQ ID NO: 180 - DNA, 21 bp, Artificial Sequence
  Synthetic oligonucleotide.
tctgstctgagatgcaatttt

SEQ ID NO: 181 - DNA, 26 bp, Artificial Sequence
  Synthetic oligonucleotide.
ggctacttcccttggtataagcaaga

SEQ ID NO: 182 - DNA, 20 bp, Artificial Sequence
  Synthetic oligonucleotide.
cagatgcaaggtcaagtgac

SEQ ID NO: 183 - DNA, 18 bp, Artificial Sequence
  Synthetic oligonucleotide.
aaggtccacagctccttc

SEQ ID NO: 184 - DNA, 21 bp, Artificial Sequence
  Synthetic oligonucleotide.
gttctggtatgtgcagtatcc

SEQ ID NO: 185 - DNA, 20 bp, Artificial Sequence
  Synthetic oligonucleotide.
agattccgtgactcaaacag

SEQ ID NO: 186 - DNA, 25 bp, Artificial Sequence
  Synthetic oligonucleotide.
agaaggtrcagcagagcccagaatc

SEQ ID NO: 187 - DNA, 17 bp, Artificial Sequence
  Synthetic oligonucleotide.
gagcrtccasgagggtg

SEQ ID NO: 188 - DNA, 18 bp, Artificial Sequence
  Synthetic oligonucleotide.
ccagtggttcaaggagtg

SEQ ID NO: 189 - DNA, 19 bp, Artificial Sequence
  Synthetic oligonucleotide.
agagaaggtcgagcaacac

SEQ ID NO: 190 - DNA, 18 bp, Artificial Sequence
  Synthetic oligonucleotide.
aagacccaagtggagcag

SEQ ID NO: 191 - DNA, 17 bp, Artificial Sequence
  Synthetic oligonucleotide.
tgacccagacagaaggc

SEQ ID NO: 192 - DNA, 16 bp, Artificial Sequence
  Synthetic oligonucleotide.
tccttggttctgcagg

SEQ ID NO: 193 - DNA, 18 bp, Artificial Sequence
  Synthetic oligonucleotide.
gcagcaggtgagacaaag

SEQ ID NO: 194 - DNA, 22 bp, Artificial Sequence
  Synthetic oligonucleotide.
cascttyttagtggagagatgg

SEQ ID NO: 195 - DNA, 20 bp, Artificial Sequence
  Synthetic oligonucleotide.
gtacaagcaaacagcaagtg

SEQ ID NO: 196 - DNA, 16 bp, Artificial Sequence
  Synthetic oligonucleotide.
cagtccgtggaccagc

SEQ ID NO: 197 - DNA, 16 bp, Artificial Sequence
  Synthetic oligonucleotide.
aacggctggagcagag

SEQ ID NO: 198 - DNA, 21 bp, Artificial Sequence
  Synthetic oligonucleotide.
gcaagttaaacaaagctctcc

SEQ ID NO: 199 - DNA, 18 bp, Artificial Sequence
  Synthetic oligonucleotide.
gtgcacttgccttgtagc

SEQ ID NO: 200 - DNA, 16 bp, Artificial Sequence
  Synthetic oligonucleotide.
ggcatcacagggaacg

SEQ ID NO: 201 - DNA, 18 bp, Artificial Sequence
  Synthetic oligonucleotide.
taccacgtggtcaagctg

SEQ ID NO: 202 - DNA, 20 bp, Artificial Sequence
  Synthetic oligonucleotide.
cagtatctaggccacaatgc

SEQ ID NO: 203 - DNA, 20 bp, Artificial Sequence
  Synthetic oligonucleotide.
cccaaagtcttacagatccc

SEQ ID NO: 204 - DNA, 18 bp, Artificial Sequence
  Synthetic oligonucleotide.
gacggctgttttccagac

SEQ ID NO: 205 - DNA, 20 bp, Artificial Sequence
  Synthetic oligonucleotide.
ggtataaacagagcgctgag

SEQ ID NO: 206 - DNA, 17 bp, Artificial Sequence
  Synthetic oligonucleotide.
ggggttgtccagtctcc

SEQ ID NO: 207 - DNA, 17 bp, Artificial Sequence
  Synthetic oligonucleotide.
gctgcagtcacccaaag

SEQ ID NO: 208 - DNA, 18 bp, Artificial Sequence
  Synthetic oligonucleotide.
gcagtcctacaggaaggg

SEQ ID NO: 209 - DNA, 19 bp, Artificial Sequence
  Synthetic oligonucleotide.
gagttacccagacacccag

SEQ ID NO: 210 - DNA, 19 bp, Artificial Sequence
  Synthetic oligonucleotide.
cctaggcacaaggtgacag

SEQ ID NO: 211 - DNA, 17 bp, Artificial Sequence
  Synthetic oligonucleotide.
gaagccaaaccaagcac

SEQ ID NO: 212 - DNA, 17 bp, Artificial Sequence
  Synthetic oligonucleotide.
gattggtcaggaagggc

SEQ ID NO: 213 - DNA, 18 bp, Artificial Sequence
  Synthetic oligonucleotide.
ggatggagtgtcaagctg

SEQ ID NO: 214 - DNA, 19 bp, Artificial Sequence
  Synthetic oligonucleotide.
ctgcagttacacagaagcc

SEQ ID NO: 215 - DNA, 19 bp, Artificial Sequence
  Synthetic oligonucleotide.
cagactccacgatacctgg

SEQ ID NO: 216 - DNA, 17 bp, Artificial Sequence
  Synthetic oligonucleotide.
ggtgaaagggcaaggac

SEQ ID NO: 217 - DNA, 17 bp, Artificial Sequence
  Synthetic oligonucleotide.
gctggaatgtggacagg

SEQ ID NO: 218 - DNA, 20 bp, Artificial Sequence
  Synthetic oligonucleotide.
ccagaaggtagcagagaccc

SEQ ID NO: 219 - DNA, 49 bp, Artificial Sequence
  Synthetic oligonucleotide.
tcgtcggcagcgtcagatgtgtataagagacagcgccactctccataag

SEQ ID NO: 220 - DNA, 48 bp, Artificial Sequence
  Synthetic oligonucleotide.
tcgtcggcagcgtcagatgtgtataagagacagcagaggbtttgaagc

SEQ ID NO: 221 - DNA, 50 bp, Artificial Sequence
  Synthetic oligonucleotide.
tcgtcggcagcgtcagatgtgtataagagacagctcctcaagtactattc

SEQ ID NO: 222 - DNA, 48 bp, Artificial Sequence
  Synthetic oligonucleotide.
tcgtcggcagcgtcagatgtgtataagagacagggccagagcctccag

SEQ ID NO: 223 - DNA, 47 bp, Artificial Sequence
  Synthetic oligonucleotide.
tcgtcggcagcgtcagatgtgtataagagacaggaaggccamgcccc

SEQ ID NO: 224 - DNA, 48 bp, Artificial Sequence
  Synthetic oligonucleotide.
tcgtcggcagcgtcagatgtgtataagagacagcaccagggaccacag

SEQ ID NO: 225 - DNA, 49 bp, Artificial Sequence
  Synthetic oligonucleotide.
tcgtcggcagcgtcagatgtgtataagagacagctcttcagggtccaga

SEQ ID NO: 226 - DNA, 51 bp, Artificial Sequence
  Synthetic oligonucleotide.
tcgtcggcagcgtcagatgtgtataagagacagtagtggagagatggtttt

SEQ ID NO: 227 - DNA, 51 bp, Artificial Sequence
  Synthetic oligonucleotide.
tcgtcggcagcgtcagatgtgtataagagacaggcaagtgggraaatagtt

SEQ ID NO: 228 - DNA, 49 bp, Artificial Sequence
  Synthetic oligonucleotide.
tcgtcggcagcgtcagatgtgtataagagacagtggaaagrgtctccac

SEQ ID NO: 229 - DNA, 51 bp, Artificial Sequence
  Synthetic oligonucleotide.
tcgtcggcagcgtcagatgtgtataagagacagaacagaaagttttcacgc

SEQ ID NO: 230 - DNA, 48 bp, Artificial Sequence
  Synthetic oligonucleotide.
tcgtcggcagcgtcagatgtgtataagagacagctycagttscttatg

SEQ ID NO: 231 - DNA, 48 bp, Artificial Sequence
  Synthetic oligonucleotide.
tcgtcggcagcgtcagatgtgtataagagacaggttsmtatggaaaga

SEQ ID NO: 232 - DNA, 50 bp, Artificial Sequence
  Synthetic oligonucleotide.
tcgtcggcagcgtcagatgtgtataagagacaggyccygcwctyctgata

SEQ ID NO: 233 - DNA, 49 bp, Artificial Sequence
  Synthetic oligonucleotide.
tcgtcggcagcgtcagatgtgtataagagacagcaacagccscacactc

SEQ ID NO: 234 - DNA, 47 bp, Artificial Sequence
  Synthetic oligonucleotide.
tcgtcggcagcgtcagatgtgtataagagacagctggrraarscccc

SEQ ID NO: 235 - DNA, 49 bp, Artificial Sequence
  Synthetic oligonucleotide.
tcgtcggcagcgtcagatgtgtataagagacagtgsgaaaggccttgag

SEQ ID NO: 236 - DNA, 50 bp, Artificial Sequence
  Synthetic oligonucleotide.
tcgtcggcagcgtcagatgtgtataagagacagcaggcaaaggtcttgtg

SEQ ID NO: 237 - DNA, 48 bp, Artificial Sequence
  Synthetic oligonucleotide.
tcgtcggcagcgtcagatgtgtataagagacagcaggcrraggccckg

SEQ ID NO: 238 - DNA, 48 bp, Artificial Sequence
  Synthetic oligonucleotide.
tcgtcggcagcgtcagatgtgtataagagacagccagggaaggtcctg

SEQ ID NO: 239 - DNA, 49 bp, Artificial Sequence
  Synthetic oligonucleotide.
tcgtcggcagcgtcagatgtgtataagagacagtgggggamgyctcatc

SEQ ID NO: 240 - DNA, 49 bp, Artificial Sequence
  Synthetic oligonucleotide.
tcgtcggcagcgtcagatgtgtataagagacagaaghctcgtcagcctg

SEQ ID NO: 241 - DNA, 48 bp, Artificial Sequence
  Synthetic oligonucleotide.
tcgtcggcagcgtcagatgtgtataagagacagaggaacaaaggagaa

SEQ ID NO: 242 - DNA, 51 bp, Artificial Sequence
  Synthetic oligonucleotide.
tcgtcggcagcgtcagatgtgtataagagacaggaagatcagtagaagatt

SEQ ID NO: 243 - DNA, 51 bp, Artificial Sequence
  Synthetic oligonucleotide.
tcgtcggcagcgtcagatgtgtataagagacaggaagagagatagaagatt

SEQ ID NO: 244 - DNA, 49 bp, Artificial Sequence; identical to SEQ ID NO: 219
  Synthetic oligonucleotide.

SEQ ID NO: 245 - DNA, 56 bp, Artificial Sequence
  Synthetic oligonucleotide.
gtctcgtgggctcggagatgtgtataagagacagcgtgatctgtcctgagaccgag

SEQ ID NO: 246 - DNA, 56 bp, Artificial Sequence
  Synthetic oligonucleotide.
gtctcgtgggctcggagatgtgtataagagacagacatcgctgtcctgagaccgag

SEQ ID NO: 247 - DNA, 56 bp, Artificial Sequence
  Synthetic oligonucleotide.
gtctcgtgggctcggagatgtgtataagagacaggcctaactgtcctgagaccgag

SEQ ID NO: 248 - DNA, 56 bp, Artificial Sequence
  Synthetic oligonucleotide.
gtctcgtgggctcggagatgtgtataagagacagtggtcactgtcctgagaccgag

SEQ ID NO: 249 - DNA, 56 bp, Artificial Sequence
  Synthetic oligonucleotide.
gtctcgtgggctcggagatgtgtataagagacagattggcctgtcctgagaccgag

SEQ ID NO: 250 - DNA, 56 bp, Artificial Sequence
  Synthetic oligonucleotide.
gtctcgtgggctcggagatgtgtataagagacaggatctgctgtcctgagaccgag

SEQ ID NO: 251 - DNA, 56 bp, Artificial Sequence
  Synthetic oligonucleotide.
gtctcgtgggctcggagatgtgtataagagacagtcaagtctgtcctgagaccgag

SEQ ID NO: 252 - DNA, 56 bp, Artificial Sequence
  Synthetic oligonucleotide.
gtctcgtgggctcggagatgtgtataagagacagctgatcctgtcctgagaccgag

SEQ ID NO: 253 - DNA, 56 bp, Artificial Sequence
  Synthetic oligonucleotide.
gtctcgtgggctcggagatgtgtataagagacagaagctactgtcctgagaccgag

SEQ ID NO: 254 - DNA, 56 bp, Artificial Sequence
  Synthetic oligonucleotide.
gtctcgtgggctcggagatgtgtataagagacaggtagccctgtcctgagaccgag

SEQ ID NO: 255 - DNA, 56 bp, Artificial Sequence
  Synthetic oligonucleotide.
gtctcgtgggctcggagatgtgtataagagacagtacaagctgtcctgagaccgag

SEQ ID NO: 256 - DNA, 56 bp, Artificial Sequence
  Synthetic oligonucleotide.
gtctcgtgggctcggagatgtgtataagagacagcactgtctgtcctgagaccgag

SEQ ID NO: 257 - DNA, 56 bp, Artificial Sequence
  Synthetic oligonucleotide.
gtctcgtgggctcggagatgtgtataagagacagttgactctgtcctgagaccgag

SEQ ID NO: 258 - DNA, 56 bp, Artificial Sequence
  Synthetic oligonucleotide.
gtctcgtgggctcggagatgtgtataagagacagggaactctgtcctgagaccgag

SEQ ID NO: 259 - DNA, 56 bp, Artificial Sequence
  Synthetic oligonucleotide.
gtctcgtgggctcggagatgtgtataagagacagtgacatctgtcctgagaccgag

SEQ ID NO: 260 - DNA, 56 bp, Artificial Sequence
  Synthetic oligonucleotide.
gtctcgtgggctcggagatgtgtataagagacagggacggctgtcctgagaccgag

SEQ ID NO: 261 - DNA, 56 bp, Artificial Sequence
  Synthetic oligonucleotide.
gtctcgtgggctcggagatgtgtataagagacagctctacctgtcctgagaccgag

SEQ ID NO: 262 - DNA, 56 bp, Artificial Sequence
  Synthetic oligonucleotide.
gtctcgtgggctcggagatgtgtataagagacaggcggacctgtcctgagaccgag

SEQ ID NO: 263 - DNA, 49 bp, Artificial Sequence
  Synthetic oligonucleotide.
tcgtcggcagcgtcagatgtgtataagagacaggctgatccattaytca

SEQ ID NO: 264 - DNA, 52 bp, Artificial Sequence
  Synthetic oligonucleotide.
tcgtcggcagcgtcagatgtgtataagagacagctgatttatatctcatacg

SEQ ID NO: 265 - DNA, 51 bp, Artificial Sequence
  Synthetic oligonucleotide.
tcgtcggcagcgtcagatgtgtataagagacagagtaatctagtattccta

SEQ ID NO: 266 - DNA, 52 bp, Artificial Sequence
  Synthetic oligonucleotide.
tcgtcggcagcgtcagatgtgtataagagacaggtttctggttaatttctac

SEQ ID NO: 267 - DNA, 52 bp, Artificial Sequence
  Synthetic oligonucleotide.
tcgtcggcagcgtcagatgtgtataagagacagttcytyattcagmattatg

SEQ ID NO: 268 - DNA, 49 bp, Artificial Sequence
  Synthetic oligonucleotide.
tcgtcggcagcgtcagatgtgtataagagacagtttcarmatcaagaag

SEQ ID NO: 269 - DNA, 52 bp, Artificial Sequence
  Synthetic oligonucleotide.
tcgtcggcagcgtcagatgtgtataagagacagcgaaatgaagaaattatgg

SEQ ID NO: 270 - DNA, 50 bp, Artificial Sequence
  Synthetic oligonucleotide.
tcgtcggcagcgtcagatgtgtataagagacaggtycctgayytactttc

SEQ ID NO: 271 - DNA, 49 bp, Artificial Sequence
  Synthetic oligonucleotide.
tcgtcggcagcgtcagatgtgtataagagacaggttgctgagctacttc

SEQ ID NO: 272 - DNA, 51 bp, Artificial Sequence
  Synthetic oligonucleotide.
tcgtcggcagcgtcagatgtgtataagagacagcttctagtttactttcgg

SEQ ID NO: 273 - DNA, 51 bp, Artificial Sequence
  Synthetic oligonucleotide.
tcgtcggcagcgtcagatgtgtataagagacagggtttactttcagaatga

SEQ ID NO: 274 - DNA, 52 bp, Artificial Sequence
  Synthetic oligonucleotide.
tcgtcggcagcgtcagatgtgtataagagacagtggcttcattctatgatac

SEQ ID NO: 275 - DNA, 54 bp, Artificial Sequence
  Synthetic oligonucleotide.
tcgtcggcagcgtcagatgtgtataagagacagtctaacttatttacagaatgg

SEQ ID NO: 276 - DNA, 49 bp, Artificial Sequence
  Synthetic oligonucleotide.
tcgtcggcagcgtcagatgtgtataagagacaggctcatgtttctctac

SEQ ID NO: 277 - DNA, 49 bp, Artificial Sequence
  Synthetic oligonucleotide.
tcgtcggcagcgtcagatgtgtataagagacagttccaaggcgcttctc

SEQ ID NO: 278 - DNA, 49 bp, Artificial Sequence
  Synthetic oligonucleotide.
tcgtcggcagcgtcagatgtgtataagagacagggacaatcagactgcc

SEQ ID NO: 279 - DNA, 49 bp, Artificial Sequence
  Synthetic oligonucleotide.
tcgtcggcagcgtcagatgtgtataagagacagggctatgatgcgtctc

SEQ ID NO: 280 - DNA, 48 bp, Artificial Sequence
  Synthetic oligonucleotide.
tcgtcggcagcgtcagatgtgtataagagacagcagggaagctgacac

SEQ ID NO: 281 - DNA, 51 bp, Artificial Sequence
  Synthetic oligonucleotide.
tcgtcggcagcgtcagatgtgtataagagacaggtttctggtgaatttcca

SEQ ID NO: 282 - DNA, 51 bp, Artificial Sequence
  Synthetic oligonucleotide.
tcgtcggcagcgtcagatgtgtataagagacagatagcactttctactgtg

SEQ ID NO: 283 - DNA, 49 bp, Artificial Sequence
  Synthetic oligonucleotide.
tcgtcggcagcgtcagatgtgtataagagacagactgttggccaggtag

SEQ ID NO: 284 - DNA, 50 bp, Artificial Sequence
  Synthetic oligonucleotide.
tcgtcggcagcgtcagatgtgtataagagacagccccagttcaatactac

SEQ ID NO: 285 - DNA, 53 bp, Artificial Sequence
  Synthetic oligonucleotide.
tcgtcggcagcgtcagatgtgtataagagacagggcttattactcaattaatg

SEQ ID NO: 286 - DNA, 48 bp, Artificial Sequence
  Synthetic oligonucleotide.
tcgtcggcagcgtcagatgtgtataagagacaggctgttcactctgcg

SEQ ID NO: 287 - DNA, 51 bp, Artificial Sequence
  Synthetic oligonucleotide.
tcgtcggcagcgtcagatgtgtataagagacagtgaaatttgtgacttctg

SEQ ID NO: 288 - DNA, 51 bp, Artificial Sequence
  Synthetic oligonucleotide.
tcgtcggcagcgtcagatgtgtataagagacagtcatggcaactgcaaatg

SEQ ID NO: 289 - DNA, 50 bp, Artificial Sequence
  Synthetic oligonucleotide.
tcgtcggcagcgtcagatgtgtataagagacaggcgccattgttcatatg

SEQ ID NO: 290 - DNA, 57 bp, Artificial Sequence
  Synthetic oligonucleotide.
gtctcgtgggctcggagatgtgtataagagacagcgtgatcaaacaaggagaccttg

SEQ ID NO: 291 - DNA, 57 bp, Artificial Sequence
  Synthetic oligonucleotide.
gtctcgtgggctcggagatgtgtataagagacagacatcgcaaacaaggagaccttg

SEQ ID NO: 292 - DNA, 57 bp, Artificial Sequence
  Synthetic oligonucleotide.
gtctcgtgggctcggagatgtgtataagagacaggcctaacaaacaaggagaccttg

SEQ ID NO: 293 - DNA, 57 bp, Artificial Sequence
  Synthetic oligonucleotide.
gtctcgtgggctcggagatgtgtataagagacagtggtcacaaacaaggagaccttg

SEQ ID NO: 294 - DNA, 57 bp, Artificial Sequence
  Synthetic oligonucleotide.
gtctcgtgggctcggagatgtgtataagagacagattggccaaacaaggagaccttg

SEQ ID NO: 295 - DNA, 57 bp, Artificial Sequence
  Synthetic oligonucleotide.
gtctcgtgggctcggagatgtgtataagagacaggatctgcaaacaaggagaccttg

SEQ ID NO: 296 - DNA, 57 bp, Artificial Sequence
  Synthetic oligonucleotide.
gtctcgtgggctcggagatgtgtataagagacagtcaagtcaaacaaggagaccttg

SEQ ID NO: 297 - DNA, 57 bp, Artificial Sequence
  Synthetic oligonucleotide.
gtctcgtgggctcggagatgtgtataagagacagctgatccaaacaaggagaccttg

SEQ ID NO: 298 - DNA, 57 bp, Artificial Sequence
  Synthetic oligonucleotide.
gtctcgtgggctcggagatgtgtataagagacagaagctacaaacaaggagaccttg

SEQ ID NO: 299 - DNA, 57 bp, Artificial Sequence
  Synthetic oligonucleotide.
gtctcgtgggctcggagatgtgtataagagacaggtagcccaaacaaggagaccttg

SEQ ID NO: 300 - DNA, 57 bp, Artificial Sequence
  Synthetic oligonucleotide.
gtctcgtgggctcggagatgtgtataagagacagtacaagcaaacaaggagaccttg

SEQ ID NO: 301 - DNA, 57 bp, Artificial Sequence
  Synthetic oligonucleotide.
gtctcgtgggctcggagatgtgtataagagacagcactgtcaaacaaggagaccttg

SEQ ID NO: 302 - DNA, 57 bp, Artificial Sequence
  Synthetic oligonucleotide.
gtctcgtgggctcggagatgtgtataagagacagttgactcaaacaaggagaccttg

SEQ ID NO: 303 - DNA, 57 bp, Artificial Sequence
  Synthetic oligonucleotide.
gtctcgtgggctcggagatgtgtataagagacagggaactcaaacaaggagaccttg

SEQ ID NO: 304 - DNA, 57 bp, Artificial Sequence
  Synthetic oligonucleotide.
gtctcgtgggctcggagatgtgtataagagacagtgacatcaaacaaggagaccttg

SEQ ID NO: 305 - DNA, 57 bp, Artificial Sequence
  Synthetic oligonucleotide.
gtctcgtgggctcggagatgtgtataagagacagggacggcaaacaaggagaccttg

SEQ ID NO: 306 - DNA, 57 bp, Artificial Sequence
  Synthetic oligonucleotide.
gtctcgtgggctcggagatgtgtataagagacagctctaccaaacaaggagaccttg

SEQ ID NO: 307 - DNA, 57 bp, Artificial Sequence
  Synthetic oligonucleotide.
gtctcgtgggctcggagatgtgtataagagacaggcggaccaaacaaggagaccttg

SEQ ID NO: 308 - DNA, 33 bp, Artificial Sequence
  Synthetic oligonucleotide.
tcgtcggcagcgtcagatgtgtataagagacag

SEQ ID NO: 309 - DNA, 34 bp, Artificial Sequence
  Synthetic oligonucleotide.
gtctcgtgggctcggagatgtgtataagagacag